SEQUENCE LISTING (8 sequences)

SEQ ID NO: 1 - DNA, 20 bp, Artificial Sequence
  SEQ ID NO 1 depicts the forward primer squence for Oct 4a gene
agccctcatttcaccaggcc

SEQ ID NO: 2 - DNA, 20 bp, Artificial Sequence
  SEQ ID NO 2 depicts the reverse primer sequence for Oct 4a gene
tgggactcctccgggttttg

SEQ ID NO: 3 - DNA, 20 bp, Artificial Sequence
  SEQ ID NO 3 depicts the forward primer sequence for LINE 1 family (79 bp)
agggacatggatgaaattgg

SEQ ID NO: 4 - DNA, 20 bp, Artificial Sequence
  SEQ ID NO 4 depicts the reverse primer sequence for LINE 1 family (79 bp)
tgagaatatgcggtgtttgg

SEQ ID NO: 5 - DNA, 21 bp, Artificial Sequence
  SEQ ID NO 5 depicts the forward primer sequence for LINE 1 family (97 bp)
tggcacatatacaccatggaa

SEQ ID NO: 6 - DNA, 23 bp, Artificial Sequence
  SEQ ID NO 6 depicts the reverse primer sequence for LINE1 family (97 bp)
tgagaatgatggtttccaatttc

SEQ ID NO: 7 - DNA, 20 bp, Artificial Sequence
  SEQ ID NO 7 depicts the forward primer sequence for LINE1 family (127 bp)
acttggaaccaacccaaatg

SEQ ID NO: 8 - DNA, 21 bp, Artificial Sequence
  SEQ ID NO 8 depicts the reverse primer sequence for LINE1 family (127 bp)
tcatccatgtccctacaaagg

(19) United States
(12) Patent Application Publication
(10) Pub. No.: US 2025/0215500 A1
(43) Pub. Date: Jul. 3, 2025

(54) METHOD FOR DETECTING MEDICAL CONDITIONS USING ANALYSIS OF VERY SMALL EMBRYONIC-LIKE STEM CELLS

(71) Applicant: 23IKIGAI PTE LTD, Singapore (SG)

(72) Inventors: Nripen SHARMA, Mumbai (IN); Ashish TRIPATHI, Mumbai (IN); Vinay Kumar TRIPATHI, Mumbai (IN); Sagar CHHABRIA, Mumbai (IN)

(21) Appl. No.: 18/681,811

(22) PCT Filed: Apr. 21, 2022
(86) PCT No.: PCT/IN2022/050380

§ 371 Date: Feb. 6, 2024

(30) Foreign Application Priority Data
Apr. 21, 2021 (IN) 202121018478

(51) Int. Cl. C12Q 1/6886; C12Q 1/6806

(52) U.S. Cl. CPC C12Q 1/6886; C12Q 1/6806; C12Q 2600/118; C12Q 2600/158; G01N 2800/52

(57) ABSTRACT

The present disclosure discloses a method for detecting a medical condition, in particular cancer, in a subject using blood sample by analyzing very small embryonic like stem cells. The present disclosure also provides with methods of detecting a medical condition or cancer in a subject by analyzing the expression of Oct4A marker in blood sample. The method as disclosed herein is simple, quick, non-invasive and specific.

### CROSS-REFERENCE

This application is a National Stage Entry of International Patent Application No. PCT/IN2022/050380, filed Apr. 21, 2022, which claims the benefit of Indian Application No. 202121018478, filed Apr. 21, 2021, each of which is incorporated by reference herein in its entirety.

### FIELD OF INVENTION

The present disclosure broadly relates to the field of healthcare technologies, and particularly provides an in-vitro method for detecting the presence or absence of a medical condition in a human subject with the aid of very small embryonic like stem cells.

### BACKGROUND OF INVENTION

Research on the genetic causes of disease has accelerated because of both the completion of the human genome and the development of the Next Generation Sequencing (NGS) techniques. The NGS techniques are available to study the molecular basis of human diseases. NGS-based approaches are also used for translating the alterations in individuals' genomes into clinically relevant information that can be used in clinical diagnostics and therapeutic, clinical decision-making strategies. These efforts have generated a large volume of potentially useful information in the form of enormous amounts of data that has boosted biomedical research. Application and interpretation of this information, however, is still cumbersome and time-consuming for researchers because the clinically relevant molecular fingerprint of the mutation profiles is derived out of tissues extracted from biopsy procedures.

Biopsy is a well-known technique which involves the removal of tissue under examination for disease diagnosis and further treatment approaches. Usually, a biopsy is an invasive technique that involves complex surgical procedures for the removal of tissue from their native environment. Tissue biopsy is the “gold standard” for cancer, but interestingly, a number of non-cancerous tissues (i.e., diseased tissues) are also excised in order to detect the origin, transmission, progression of disease etc., that dilutes the original disease data and leads to false positives including misdiagnosis. Almost all tissues can be studied through biopsy including muscle, thyroid, bladder, heart, prostate, skin, lung, lymph node, liver, kidney, nerves etc. Some diseases for which biopsies are included in the scientific literature are cortical demyelination in brain white matter lesions for early detection of multiple sclerosis (Lucchinetti et. al. 2011), percutaneous renal biopsy for kidney diseases, cirrhotic liver disease, hepatitis C-associated glomerulonephritis and cryoglobulinemic vasculitis, monoclonal gammopathy etc. (Hogan, Mocanu, and Berns 2016), synovial biopsy for detection of mononuclear infiltrates, fibrosis, angiogenesis, macrophage infiltration and lining layer thickening in tissues of osteoarthritis patients (Ene et al. 2015), shave, punch or incisional biopsy for inflammatory skin disorders (Harvey, Chan, and Wood 2017), computer-tomography guided lung biopsy for evaluation of COPD (Asai et al. 2013), myocardial biopsy (Francis and Lewis 2018), liver biopsy for cirrhotic patients (Sherman et al. 2007), etc.

The Patent document WO2011143361A2 discloses a composition, kits and a method for molecular profiling for diagnosing thyroid cancer and other cancer. For the purpose of carrying out the method, the surgical biopsy is used for collecting the thyroid tissue sample.

Although the conventional methods mainly use surgical biopsy to identify a medical condition in a subject, most tissue biopsies result in surgical complications, bleeding, and adverse side-effects etc., and hence are not recommended as opposed to biofluid tests such as of blood, urine, saliva etc. Tissue biopsies are difficult to perform, resulting in painful, often discomfort procedures that may not identify the exact anatomical location of the tumor or may further cause metastasis-promoting complications due to surgical excision of angiogenesis-rich areas. Owing to the complexities of the tissue biopsy procedure and mixed results obtained, and the lack of clarity associated with such studies with respect to the tissue to be studied vis-a-vis the condition of a subject, there is a knowledge gap which exists in this area of work.

Stem cells, particularly of embryonic origin, possess pluripotency markers viz. Oct4, Nanog, Sox2 and their isoforms are indicative of varied differentiation potentials into multiple tissues forming organs in development, homeostasis and aging. Since stem cells contribute to tissue development, they act as molecular biosensors implicative of tissue damage and injury, a hallmark of medical conditions.

Thus, there is a dire need in the art to deploy non-invasive methods based on analyzing the expression of stem cell markers, as stem cell marker are prominent biomarkers for determining severity of medical conditions and identification of embryonic-like stem cell markers in body fluids can detect medical condition non-invasively.

### SUMMARY OF INVENTION

In an aspect of the present disclosure, there is provided an in-vitro method for detecting a medical condition in a subject, said method comprising: (a) obtaining a blood sample and adding a salt solution to the blood sample; (b) layering the blood sample of step (a) over a neutral buffer and subjecting the blood sample to a density gradient centrifugation at a speed in the range of 200-900 g to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) subjecting the second pellet to a lysis for obtaining nucleic acid from the enriched very small embryonic like stem cells; (f) performing an assay with the nucleic acid of step (e) for analysing expression level of Oct 4A in the very small embryonic like stem cells; and (g) comparing the expression level of Oct 4A in the very small embryonic like stem cells from the blood sample with an expression level of Oct 4A in a control sample for determining an increase or decrease in the expression level, wherein an increase in the range of 1.1-3 folds in the expression level of Oct 4A in the very small embryonic like stem cells from the blood sample as compared to the expression level of Oct 4A in a control sample detects the presence of a medical condition in the subject.

In another aspect of the present disclosure, there is provided an in-vitro method for predicting onset of cancer or predicting the presence of tumor or cancer in a subject, said method comprising: (a) obtaining a blood sample and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) subjecting the second pellet to a lysis for obtaining nucleic acid from the enriched very small embryonic like stem cells; (f) performing an assay with the nucleic acid of step (e) for analyzing expression level of Oct 4A in the very small embryonic like stem cells; and (g) comparing the expression level of Oct 4A in the very small embryonic like stem cells from the blood sample with an expression level of Oct 4A in a control sample for detecting an increase or decrease in the expression level, wherein an increase in the range of 3-5 folds in the expression level of Oct 4A in the very small embryonic like stem cells from the blood sample as compared to the expression level of Oct 4A in control sample predicts the onset of cancer in the subject, wherein an increase of at least 5 folds in the expression level of Oct 4A in the very small embryonic like stem cells from the blood sample as compared to the expression level of Oct 4A in the control sample detects the presence of tumor or cancer in the subject.

In another aspect of the present disclosure, there is provided a method for detecting presence of a medical condition in a subject, said method comprising: (a) obtaining a blood sample from a subject and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) enumerating the number of very small embryonic like stem cells in the second pellet; and (f) comparing the number of very small embryonic like stem cells in the blood sample with the number of very small embryonic like stem cells in a control blood sample, wherein an increase in the number of very small embryonic like stem cells in the blood sample by at least two folds as compared to the number of very small embryonic like stem cells in a control blood sample detects the presence of a medical condition in the subject.

In another aspect of the present disclosure, there is provided a method for detecting presence of tumor or cancer or onset of cancer in a subject, said method comprising: (a) obtaining a blood sample from a subject and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 200-4000 g to obtain a second pellet comprising enriched very small embryonic like stem cells; (e) enumerating the number of very small embryonic like stem cells in the second pellet; and (f) comparing the number of very small embryonic like stem cells in the blood sample with the number of very small embryonic like stem cells in a control blood sample for detecting an increase or decrease in the number of very small embryonic like stem cells, wherein an increase in the number of very small embryonic like stem cells in the blood sample in the range of 2-5 folds as compared to the number of very small embryonic like stem cells in a control blood sample detects the onset of cancer in the subject; and wherein an increase in the number of very small embryonic like stem cells in the blood sample by at least 5 folds as compared to the number of very small embryonic like stem cells in a control blood sample detects the presence of tumor or cancer in the subject.

In another aspect of the present disclosure, there is provided an in-vitro method for detecting a positive response to anti-cancer therapy, said method comprising: (a) obtaining a blood sample-I before administration of an anti-cancer therapy; (b) enriching very small embryonic like stem cells from the blood sample-I by a process comprising: (bi) adding a salt solution to the blood sample I (bii) layering the blood sample-I of step (bi) over a neutral buffer and subjecting the blood sample to a density gradient centrifugation at a speed in the range of 200-900 g, to obtain a first pellet comprising red blood cells (RBC); (biii) lysing the RBC in the first pellet to obtain a RBC-lysed solution; (biv) centrifuging the RBC-lysed solution at a speed in the range of 400-4000 g to obtain a second pellet comprising enriched very small embryonic like stem cells from the blood sample-I; (c) obtaining a blood sample-II after at least 2 weeks of administration of the anti-cancer therapy; (d) enriching very small embryonic like stem cells from the blood sample-II by a process comprising: (di) adding a salt solution to the blood sample II, (dii) layering the blood sample-II of step (di) over a neutral buffer and subjecting the blood sample to a density gradient centrifugation at a speed in the range of 200-900 g, to obtain a first pellet comprising red blood cells (RBC); (diii) lysing the RBC in the first pellet to obtain a RBC-lysed solution; (div) centrifuging the RBC-lysed solution at a speed in the range of 400-4000 g to obtain a second pellet comprising enriched very small embryonic like stem cells from the blood sample-II; (e) obtaining nucleic acid-I from the enriched very small embryonic like stem cells from the blood sample-I; (f) obtaining nucleic acid-II from the enriched very small embryonic like stem cells from the blood sample-II; (g) independently performing an assay with the nucleic acid-I and the nucleic acid-II for analysing expression level of Oct 4A; and (h) comparing the expression levels of Oct 4A from the nucleic acid-II with the expression level of Oct 4A from the nucleic acid-I for detecting an increase or decrease in the expression level, wherein a decrease in the expression level of Oct 4A from the nucleic acid-II as compared to the expression level of Oct 4A from the nucleic acid-I detects a positive response to the anti-cancer therapy.

In another aspect of the present disclosure, there is provided an in-vitro method for detecting cancer, said method comprising: (a) obtaining a blood sample and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) subjecting the second pellet to a lysis for obtaining nucleic acid from the enriched very small embryonic like stem cells; (f) performing an assay with the nucleic acid for analysing expression level of Oct 4A in very small embryonic like stem cells; (g) comparing the expression level of Oct 4A in very small embryonic like stem cells in the sample with an expression level of Oct 4A in very small embryonic like stem cells in a control sample for detecting an increase or decrease in the expression level, wherein an increase in the expression level of Oct 4A in very small embryonic like stem cells in the sample by at least 5 folds as compared to the expression level of Oct 4A in very small embryonic like stem cells in the control sample indicates presence of cancer; and (h) performing sequence-based assays on the nucleic acid and analyzing for mutation in at least one cancer-related marker, wherein presence of mutation in the at least one cancer-related marker indicates presence of a specific type of cancer based on the cancer-related marker analysed.

In another aspect of the present disclosure, there is provided an in-vitro method for treating cancer, said method comprising: (a) obtaining a blood sample from a subject and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) subjecting the second pellet to a lysis for obtaining nucleic acid from the enriched very small embryonic like stem cells; (f) performing an assay with the nucleic acid of step (e) for analysing expression level of Oct 4A in the very small embryonic like stem cells; (g) comparing the expression level of Oct 4A in very small embryonic like stem cells in the sample with an expression level of Oct 4A in a control sample for determining an increase or decrease in the expression level, wherein an increase in the expression level of Oct 4A in very small embryonic like stem cells in the sample by at least 5 folds as compared to the expression level of Oct 4A in the control sample detects cancer; and (h) administering anti-cancer therapy to the subject for treating cancer, wherein the administration of anti-cancer therapy decreases the expression level of Oct 4A in the subject.

In another aspect of the present disclosure, there is provided a method for detecting the presence of a medical condition in a subject, said method comprising: (a) obtaining a blood sample from a subject and diluting the sample with a salt solution; (b) enumerating the number of very small embryonic like stem cells in the blood sample; and (c) comparing the number of very small embryonic like stem cells in the blood sample with the number of very small embryonic like stem cells in a control blood sample for detecting an increase or decrease in the number of very small embryonic like stem cells, wherein an increase in the number of very small embryonic like stem cells in the blood sample by at least 2 folds as compared to the number of very small embryonic like stem cells in a control blood sample detects the presence of a medical condition in the subject.

In another aspect of the present disclosure, a method for predicting the onset of cancer or detecting presence of tumor or cancer in a subject, said method comprising: (a) obtaining a blood sample from a subject and diluting the sample with a salt solution (b) enumerating the number of very small embryonic like stem cells in the blood sample; and (c) comparing the number of very small embryonic like stem cells in the blood sample with the number of very small embryonic like stem cells in a control blood sample for detecting an increase or decrease in the number of very small embryonic like stem cells, wherein an increase in the number of very small embryonic like stem cells in the blood sample in the range of 2-5 folds as compared to the number of very small embryonic like stem cells in a control blood sample predicts the onset of cancer in the subject, and wherein an increase in the number of very small embryonic like stem cells in the blood sample by at least 5 folds as compared to the number of very small embryonic like stem cells in a control blood sample detects the presence of tumor or cancer in the subject.

In another aspect of the present disclosure, a method for detecting the presence of a medical condition in a subject, said method comprising: (a) enumerating the number of very small embryonic like stem cells in vivo in blood of a subject; and (b) comparing the number of very small embryonic like stem cells in the blood of the subject with the number of very small embryonic like stem cells in a control blood sample for detecting an increase or decrease in the number of very small embryonic like stem cells, wherein an increase in the number of very small embryonic like stem cells in the blood of the subject by at least 2 folds as compared to the number of very small embryonic like stem cells in the control blood sample detects the presence of a medical condition in the subject.

In another aspect of the present disclosure, a method for predicting the onset of cancer or presence of tumor or cancer in a subject, said method comprising: (a) enumerating the number of very small embryonic like stem cells in vivo in blood of a subject; and (b) comparing the number of very small embryonic like stem cells in the blood of the subject with the number of very small embryonic like stem cells in a control blood sample for detecting an increase or decrease in the number of very small embryonic like stem cells, wherein an increase in the number of very small embryonic like stem cells in the blood of the subject in the range of 2-5 folds as compared to the number of very small embryonic like stem cells in the control blood sample predicts the onset of cancer in the subject, and wherein an increase in the number of very small embryonic like stem cells in the blood of the subject by at least 5 folds as compared to the number of very small embryonic like stem cells in the control blood sample predicts the presence of tumor or cancer in the subject.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the HrC scale (scale correlating the expression of Oct 4A from VSELs to the medical condition) showing different ranges which were found to correlate with different stages of cancer, in accordance with an implementation of the present disclosure.

FIG. 2 depicts the distribution of types of cancer patients enrolled in the study, in accordance with an implementation of the present disclosure.

FIG. 3 depicts a pie chart showing distribution of subjects identified as non-cancer (green), inflammation & high risk (dark yellow), Stage I cancer (pink), stage II cancer (red), stage III cancer (maroon red) and stage IV cancer (purple) on the basis of their HrC score, in accordance with an implementation of the present disclosure.

FIG. 4 depicts a performance assessment of HrC test based on statistical analysis. Dot plot values correspond to 1,000 patient sample points as per data of clinical study participants. All the figures were plotted using R package via ggplot library, in accordance with an implementation of the present disclosure.

FIG. 5 depicts the representative infographic image summarizes the process of clinical study screening, recruitment, distribution, analysis, and interpretation. Representative data obtained in the study by studying the study subjects and classifying based on the HrC values. Graph represents the distribution of subjects aligned on the basis of their HrC values in ascending order. They were identified as non-cancer (green), inflammation & high risk (dark yellow), Stage I cancer (pink), stage II cancer (red), stage III cancer (maroon red) and stage IV cancer (purple), in accordance with an implementation of the present disclosure.

FIG. 6 depicts the distribution of subjects aligned on the basis of their HrC values arranged in ascending order and identified as non-cancer, Inflammation, high risk and Stage I cancer, in accordance with an implementation of the present disclosure.

FIG. 7 depicts the distribution of subjects aligned on the basis of their HrC values arranged in ascending order and identified as non-cancer and stage II cancer, in accordance with an implementation of the present disclosure.

FIG. 8 depicts the distribution of subjects aligned on the basis of their HrC values arranged in ascending order and identified as non-cancer and stage III cancer, in accordance with an implementation of the present disclosure.

FIG. 9 depicts the distribution of subjects aligned on the basis of their HrC values arranged in ascending order and identified as non-cancer and stage IV cancer, in accordance with an implementation of the present disclosure.

FIG. 10 depicts the comparative analysis of the number of very small embryonic like stem cells (VSELs) obtained from the blood of a healthy subject and a cancer patient, in accordance with an implementation of the present disclosure.

FIG. 11 depicts the modalities for quantifying the VSELs in a subject in-vivo for correlating it with a medical condition of the subject, in accordance with an implementation of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “control sample” refers to a blood sample from a healthy subject. The control sample is to refer to VSELs obtained from the respective sample in order to enable the comparison of Oct 4A expression level of VSELs obtained from a sample with the VSELs obtained from a control sample.

The term “medical condition” includes all disorders, lesions, diseases, injury, genetic or congenital, or a biological or psychological condition that lies outside the range of normal, age-appropriate human variation.

The term “cancer” refers to the physiological condition in mammals that is characterized by unregulated cell growth. The term “cancer” as used in the present disclosure is intended to include benign, malignant cancers, dormant tumors, or micrometastasis. The types of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and Islet cell cancer), mesothelioma, schwannoma (including acoustic neuroma), meningioma, adenocarcinoma, melanoma, and leukemia or lymphoid malignancies. More particular examples of cancers include breast cancer, liver cancer, ovarian cancer, lung cancer, leukemia, prostate cancer, lymphoma, pancreatic cancer, cervical cancer, colon cancer, osteosarcoma, testicular cancer, thyroid cancer, gastric cancer, Ewing sarcoma, bladder cancer, gastrointestinal stromal tumor (GIST), kidney cancer (e.g., renal cell carcinoma), squamous cell cancer (e.g. epithelial squamous cell cancer), lung cancer (including small-cell lung cancer (SCLC), non-small cell lung cancer (NSCLC), adenocarcinoma of the lung, and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, hepatoma, breast cancer (including metastatic breast cancer), bladder cancer, colon cancer, rectal cancer, colorectal cancer, endometrial or uterine carcinoma, salivary gland carcinoma, prostate cancer, vulval cancer, thyroid cancer, hepatic carcinoma, anal carcinoma, penile carcinoma, Merkel cell cancer, mycoses fungoids, testicular cancer, esophageal cancer, tumors of the biliary tract, head and neck cancer, as well as B-cell lymphoma (including low grade/follicular non-Hodgkin's lymphoma (NHL); small lymphocytic (SL) NHL; intermediate grade/follicular NHL; inter mediate grade diffuse NHL; high grade immunoblastic NHL; high grade lymphoblastic NHL; high grade small non-cleaved cell NHL; bulky disease NHL, mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The term “detects” or “detection” refers to a detection which has been performed outside of a living patient using a sample from the patient.

The term “predicts” or “prediction” refers to an action of knowing something that will happen in future or in due course of time.

The term “blood sample” refers to the whole blood sample that is obtained from a subject. The scope of the method as disclosed herein begins from the stage of having obtained the blood sample, the method does not involve any invasive techniques, neither does it involve operating upon a subject. The term “blood sample” encompasses to include any form of processed blood sample also. By processing, the present disclosure intends to cover any method for enriching a specific population of cells or a mere processing so as to enable the blood sample to be used for testing by “in-vitro” methods.

The term “in-vitro” refers to a task or method or experiment being performed or taking place in a test tube, culture dish, or elsewhere outside a living organism.

The term “very small embryonic-like stem cell” or “VSELs” refers to a cell which is a type of pluripotent stem cell that is well-known in the art. The VSELs as per the present disclosure, are lesser than 7 microns in size.

The term “biomarker” refers to a biomolecule that is a nucleic acid and is used to characterize a particular cell population. The term is intended to cover both DNA and RNA forms of nucleic acid. The term “biomarker of very small embryonic-like stem cell” refers to any biomarker which can be used to characterize a population of VSELs.

The term “subject” refers to any mammal whose blood sample has been taken for analysis using the in-vitro method of the present disclosure. The exemplification is based on humans used as subjects.

The term “image analysis” refers to any imaging technology, both invasive and non-invasive, utilized to enumerate the number of VSELs population in blood of subjects to detect presence or absence of cancer and stage of cancer. The image analysis may also assist in identifying the presence or absence of a medical condition in a subject.

The term “invasive” refers to any technique that involves entry into the living body as by way of incision or by way of insertion of an instrument.

The term “body fluid” refers to any fluid secretion from a human body. It refers to blood, or sputum, or urine, or any other types of fluid from the human body.

Cancer-related marker comprises all the well-known cancer-related markers in the field of cancer study as per the scientific literature. A non-limiting list of cancer-related marker is mentioned herewith, ABL1, EVI1, MYC, APC, IL2, TNFAIP3, ABL2, EWSRI, MYCL1, ARHGEF12, JAK2, TP53, AKT1, FEY, MYCN, ATM, MAP2K4, TSC1, AKT2, FGFR1, NCOA4, BCL11B, MDM4, TSC2, ATF1, FGFR1OP, NFKB2, BLM, MEN1, VHL, BCL11A, FGFR2, NRAS, BMPRIA, MLH1, WRN, BCL2, FUS, NTRK1, BRCA1, MSH2, WT1, BCL3, GOLGA5, NUP214, BRCA2, NF1, BCL6, GOPC, PAX8, CARS, NF2, BCR, HMGA1, PDGFB, CBFA2T3, NOTCH1, BRAF, HMGA2, PIK3CA, CDHI, NPML, CARD11, HRAS, PIML, CDH11, NR4A3, CBLB, IRF4, PLAGI, CDK6, NUP98, CBLC, JUN, PPARG, CDKN2C, PALB2, CCND1, KIT, PTPN11, CEBPA, PML, CCND2, KRAS, RAF1, CHEK2, PTEN, CCND3, LCK, REL, CREB1, RB1, CDX2, LM02, RET, CREBBP, RUNX1, CTNNB1, MAF, ROS1, CYLD, SDHB, DDB2, MAFB, SMO, DDX5, SDHD, DDIT3, MAML2, SS 1 8, EXT1, SMARCA4, DDX6, MDM2, TCLIA, EXT2, SMARCB1, DEK, MET, TET2, FBXW7, SOCS1, EGFR, MITF, TFG, FH, STK11, ELK4, MLL, TLX1, FLT3, SUFU, ERBB2, MPL, TPR, FOXP1, SUZ12, ETV4, MYB, USP6, GPC3, SYK, ETV6, IDH1, TCF34A, and combinations thereof. Similarly, a list of non-limiting genes comprises all the medical-condition related markers in the field of the disease study as per the scientific literature.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific implementations described herein, which are intended for the purposes of exemplification only. Functionally-equivalent products, compositions, and methods are clearly within the scope of the disclosure, as described herein. Cancer is only one aspect of the medical condition as per the present disclosure since as an example it is widely studied, but the invention pertains to all medical conditions.

Cancer is associated with mutated genes, and analysis of tumour-linked genetic alterations is increasingly used for diagnostic, prognostic, and treatment purposes. In the past decade, ‘personalized’ or ‘stratified’ management based on the molecular features of tumours of patients has entered routine clinical practice. The genetic profile of solid tumours is currently obtained from surgical or biopsy specimens; however, the procedure cannot always be performed routinely owing to its invasive nature. First, a comprehensive characterization of multiple tumor specimens obtained from the same patient has illustrated that intratumor heterogeneity exists between different regions in the same tumor (spatial heterogeneity), as well as between the primary tumor and local or distant recurrences in the same patient (temporal heterogeneity) (Gerlinger et al. 2012). Moreover, recent studies have characterized the dynamic changes of tumor features over time with the emergence of treatment-resistant subclones that were present at a minor frequency in the primary tumor (Bedard et al. 2013). Thus, inter- and intratumor heterogeneity poses a pivotal challenge to guide clinical decision-making in oncology as biopsies may be inaccurate in capturing the complete genomic landscape of a patient's tumour (Bedard et al. 2013). Second, the complete ‘picture’ of the tumor is often limited by the tumor accessibility because of the increased rate of clinical complications associated with the invasive procedures necessary to obtain tissue at the time of initial diagnosis as well as throughout the course of disease treatment (Mlika et al. 2016). The poor performance status of many advanced cancer patients may also limit the role of uncomfortable interventional biopsy procedures (Mlika et al. 2016). Moreover, a significant barrier to biomarker testing is the availability of an adequate amount of tissue (e.g., tumor cellularity and size of the specimen) due to increasing diagnostic demands and declining amounts of tissue delivered per patient. Up to 80% of cancer patients with advanced disease will only have tissue from small biopsies or cytology, limiting the ability to perform additional tests, and as many as 31% of patients do not have accessible tissue (Wong et al. 2014). Even when tissue can be collected, preservation methods such as formalin fixation can display high levels of C>T/G>A transitions in the 1-25% allele frequency range, potentially leading to false-positive results for molecular assays (Wong et al. 2014). Finally, tissue biopsies also increase the cost of patient care, and the turnaround time for getting results can sometimes be longer than those expected by the physician for patient treatment. In light of these limitations on the use of tissue biopsies, new ways to observe tumor genetics and tumor dynamics is the need of the hour.

More recently, DNA methylation-based detection of CpG residues in circulating free DNA has been identified as universal biomarkers of common cancers as well as other diseases such as neurodegeneration and psychiatric disorders. However, some disadvantages of DNA methylation-based detection techniques are (1) time consuming and lengthy procedure, (2) relatively expensive technique, (3) detection highly dependent on assay conditions and presence of CpG residues at specific DNA restriction sites, (4) requires large amounts of DNA which is virtually absent at earlier stages of disease and (5) early-screening sensitivity is very low especially at stage I of detection which is a critical stage for prevention of cancer progression.

The three criteria for an ideal cancer detection diagnostic tool are: (i) sensitivity, ability to correctly detect the disease accurately (ii) specificity, ability to distinguish healthy, non-cancerous individuals and (iii) localization or classification, ability of test to pinpoint the type of cancer and its tissue of origin. Currently, the most studied blood-based non-invasive tests for cancer detection utilize circulating tumor cells (CTCs), circulating tumor DNA (ctDNA) and exosomes based on identification of mutations and expression of cancer-specific biomarkers (Zhou et al., 2020). Even though CTCs and ctDNA can be considered as an attractive tool for early detection and diagnosis of cancer, several studies have questioned the sensitivity, specificity of these tests for cancer prognosis (Kowalik et al., 2017).

Circulating tumor cells and tumor DNA that slip into blood circulation from dying cancer cells (by necrosis) in patients can be detected, and advanced technologies have been developed to identify even a single molecule of tumor DNA including genetic mutations/DNA methylation patterns in bloodstream (Killock 2018). However, not all early-stage tumors shed DNA and CTCs and hence it is not possible to accurately depict the molecular signature of cancer unless a novel approach is pursued. Moreover, co-morbid inflammatory diseases might shed DNA (Chaudhary and Mittra, 2019) that will conflict with cancer detection for accurate disease diagnosis, thus, compromising both sensitivity and specificity. Cancer stem cells (CSCs) on the other hand are rare and difficult to isolate and may not accurately depict the stages of cancer. Overall, there is a need for a highly, accurate, non-invasive blood-based monitoring system to detect cancer of various stages and sub-types.

Oct4, Nanog and Sox2 are critical stem cell pluripotency markers that are expressed in blood and cancerous tissues (Wang and Herlyn, 2015; Monfener et al., 2019) and depict the disease prognosis, rate of survival, effect of chemotherapy and other such disease-related parameters. Thus, developing a highly, specific and sensitive prognostic “liquid biopsy” tool that will enable clinicians to identify if cancer is present, cancer is imminent as well as the stage of cancer. However, though there are adequate citations, circulating tumor cells and cancer stem cells are present in rare quantities in blood and tissue biopsies and the isolation is cumbersome also. Hence, there is a need to measure the levels of these biomarkers in normal cells of blood such as hematopoietic stem cells, mesenchymal stem cells etc. In fact, these markers have been tested for in blood samples of all patients in a recent study (Sodja et al 2016), however, correlation with stage of cancer has not been investigated.

Very small embryonic like stem cells (VSELs), are primitive stem cells found in numerous tissues and possess pluripotent properties i.e. ability to differentiate into multiple cell types/tissues. VSELs, are quiescent in nature, but, under oncogenic stress, are activated and have the ability to differentiate into cancer stem cells or tumor initiating cells. These cells subsequently lead to cancer initiation, progression and metastasis. However, both cancer cells and VSELs possess Oct4A as a common marker, and the overexpression of this marker is associated with metastasis and invasiveness. Thus, the present disclosure discloses Oct4A from VSELs as a marker for early detection (or absence of cancer) as well as grading of cancer as per stages (I, II, III, IV) of cancer. The present disclosure discloses a mathematical scale, termed as HrC scale, that is proportional numerically to the different stages of cancer as per range of values indicated herein.

The method as per the present disclosure comprises isolating VSELs from blood and utilizing the isolated VSELs/enriched VSELs as a diagnostic tool for detecting cancer/tumour, onset of cancer or detecting any medical condition. Based on Oct4A levels in VSELs isolated from the blood, the method is able to correlate the expression of Oct4A with not only the presence or absence of cancer but also the stage of cancer in a large variety of cancers including solid tumors, haematological malignancies and sarcomas that led to development of a mathematical scale termed as HrC. The HrC scale links VSEL Oct4A expression with cancer based on scoring of 0-2: indicative of absence of cancer/inflammation, 2-6 (refers to 1.1-3 fold change in the expression level of Oct 4A): inflammatory status indicative of medical conditions such as diabetes, tuberculosis, Alzheimer's disease, dementia, cardiovascular disease, arthritis, etc., 6-10 (refers to 3-5 fold change in the expression level of Oct 4A): category includes subjects which are at imminent threat of developing cancer, 10-20 (refers to 5-10 fold change in the expression level of Oct 4A): stage I cancer, 20-30 (refers to 10-15 fold change in the expression level of Oct 4A): stage II cancer, 30-40 (refers to 15-20 fold change in the expression level of Oct 4A): stage III cancer and >40 (refers to more than 20 fold change in the expression level of Oct 4A): stage IV cancer. Therefore, the method as per the present disclosure comprises isolating VSELs from blood and correlating its Oct4A expression with staging of cancer leading to the development of a powerful diagnostic and prognostic tool. Also, Oct4A measurement from VSELs has been shown to effectively diagnose the effect of oncotherapy, disease-free survival and recurrence rate with 100% specificity and sensitivity.

The present disclosure provides the significant advantages over tumor cell-mediated cancer detection systems as follows: (1) current “liquid biopsy” diagnostic tools are limited by their sensitivity and specificity, possibly because they are derived from circulating tumor cells, cell free DNA, adult stem cells etc. and a diverse set of biomarkers or DNA methylation profiles are investigated rather than pluripotent stem cells and their markers, (2) rather than known therapeutic utilization of VSELs for regenerative medicine, diagnostic use of VSELs can be made based on blood using a validated HrC scaling system, (3) VSELs can be isolated from 1 ml of blood and hence it has superior advantage as opposed to circulating tumor cells, cell free DNA etc. that require larger volumes for detection, (4) Oct4A measurement is exclusive to enriched VSELs from 1 ml of blood, (5) VSELs based Oct4A measurement is from normal cells indicative of cancer (due to its pluripotency and oncogenic properties) as compared to circulating tumor cells (that may not be prevalent in all tumor types) and cell free DNA (that may not be tumor derived and heterogeneous in nature) and (6) VSELs Oct4A measurement is clinically useful not only to detect presence of a significant variety of cancers (solid tumors, hematologic malignancies and sarcomas), but also imminent cancer before tumor formation, stages of cancer, benign vs. malignant phenotype, inflammatory state, effect of oncotherapy, relapse rate etc. Specifically, the presence of a particular stage of cancer (I, II, III or IV) can assist doctors in decision making for stage-specific therapeutic treatment modalities and non-invasive detection of cancer and its progression. Similarly, imminent cancer detection can lead to preventative strategies while HrC scale testing after oncotherapy can help determine disease survival rate, effect of treatment and probability of recurrence. Thus, Oct4A, an oncogene, is described as the first pluripotent marker that can detect cancer and its stages with 100% sensitivity and specificity as per a trial of 500 non-cancer and 500 cancer patients. Mechanistically, this is primarily due to its constitutive activation in VSELs, defining its pluripotency, and hence the clinical manifestations of a) VSELs initiating cancer endogenously, b) VSELs transformation to cancer stem cells by yet unknown mechanisms, c) cancer stem cells as major drivers of malignancy, as well as invasiveness, migration and motility, d) detection of enriched VSELs in blood and e) Oct4A overexpression as an exclusive marker of primitive and malignant cell phenotype.

In order to overcome the problems associated with known techniques, the present disclosure discloses a simple and non-invasive technique for identifying a medical condition and inflammatory status in a human subject, particularly presence or absence of cancer and its stages. As per the method, a blood or a urine sample is sufficient enough to obtain details equivalent to those obtained after performing an invasive traditional biopsy technique. Further, the method of the present disclosure clearly pin-point the medical condition, which has not even shown any symptoms in a human subject, thus, allowing sufficient time for a medical practitioner to treat the human subject. The method of the present disclosure involves enriching very small embryonic-like stem cells (VSELs) from a sample (blood), isolating nucleic acid from the enriched very small embryonic-like stem cells. Such nucleic acid can represent the whole genome and/or transcriptome and/or exome and/or mitochondrial genome/transcriptome/exome of the human subject. The nucleic acid thus obtained is subjected to the sequence analysis by using Next Generation Sequencing or similar techniques to obtain a sequence profile. The profile is compared with a reference sequence to check for the presence of any mutation in at least one marker, wherein the presence of the mutation identifies the presence of a medical condition in the subject. The VSELs as per the present disclosure is positive for certain biomarkers of VSELs as described herein. The markers can be well-known markers specific for any tissue for which the medical condition has to be identified. Biopsies can give vast variance in expressions and mutations depending on which spot the biopsy is done in a tissue. However, the method as disclosed herein, applies at the point of mutation formation, tissue-specific gene expression, and hence removes heterogeneity. As per the present method, the genome and transcriptome data received from the sample of a human subject comprising of 50,000-100,000 expression profiles is fed to an algorithm, which in turn gives us RNA information at a tissue level of organs in the body from a blood or a urine sample. The mutation and expression data will be cross-referenced with the scientific literature and human transcriptome/gene expression databases to identify a set of genes associated with a medical condition. The algorithm can connect transcriptome and whole-genome data to generate readings for tissue-level transcriptome data. Furthermore, based on the data, the organ parameters such as its functional activity, indicators of inflammation, oxidative stress, biological pathways, molecular mechanisms, mitochondrial metabolism, etc. would also be identified. Based on the identification of primary and secondary organs associated with the transcriptome and mutation data using the algorithm, delineating the susceptibility to a variety of human diseases would also be possible. Further, the method described in the present disclosure also enables testing for rare diseases such as and not limiting to spinal muscular dystrophy, Ehlers-Danlos syndrome, Proteus syndrome, sickle cell anemia, Hutchinson-Gilford progeria, etc. that are the end result of genetic mutations. The method as described in the present disclosure, is capable of enriching VSELs in peripheral blood samples, that can be characterized by the presence of Oct4A, Fragilis, and Stella biomarkers. Once the identity of the VSELs is established, the expression levels of the biomarkers such as Oct4A, Fragilis, and Stella is compared to the expression in a control sample, wherein an increase in the expression level of the VSELs biomarkers as compared to the control indicates presence or absence of medical condition and the presence of an inflammatory condition in the human subject. Further, performing the sequencing of the nucleic acid obtained from VSELs is capable of providing deep insights into molecular mechanisms and biological pathways that corroborate the detection. Also, presence or absence of mutation in specific markers identifies the underlying medical condition in the subject. As an alternate implementation of the present disclosure, the protein levels in the enriched VSELs can also be measured to analyse the protein levels of Oct 4A in the VSELs obtained from the sample of a human subject. The increase in folds of Oct 4A protein can be correlated to the presence or absence of cancer. The protein levels can also be correlated to the staging of cancer. Further, the protein levels can also be correlated to the presence or absence of a medical condition in the subject. As per one of the implementations of the present disclosure, the blood from a subject is obtained by a pin-prick (1 ml, or 2 ml, or 5 ml, or 10 ml or 20 ml blood). Following the blood collection, the protein level of Oct 4A is estimated by using an automated ELISA kit, automated immunofluorescence assay kits within minutes to hours in a high-throughput manner. The level of Oct 4A in a sample is correlated with the level of Oct 4A in a control sample (healthy subject), wherein an increase in the protein level of Oct 4A is indicative of presence of a medical condition, or prediction of imminent cancer, or presence of cancer. The comparison of the protein levels of Oct 4A can further indicate the stage/grade of cancer. As per another implementation of the present disclosure, the blood from the subject is processed in a biosafety level II facility automatically using a centrifuge to isolate the VSELs from the pellet. The automation is further extended to isolate DNA and RNA, test its quality, and determine Oct 4A expression using RT-PCR (to determine the stage, grade of cancer, tumor load, size and differentiation) or whole genome sequencing using NGS (to determine the primary and secondary site of cancer).

In order to summarise, the method of the present disclosure is able to provide the genetic blueprint of the human subject by analysing the nucleic acid obtained from VSELs isolated from a blood sample of the human subject. The increase in the expression of Oct 4A, or Stella, or Fragilis in the blood sample of the human subject as compared to a control sample indicates an underlying medical condition and also indicates the inflammatory status in the human subject. The underlying medical condition is accurately pin-pointed by analysing the nucleic acid obtained from VSELs for the presence or absence of mutation in the specific markers. Thus, effectively, providing data equivalent to that of a biopsy, merely from a blood sample.

As per the present disclosure, any known marker can be analysed from the sequence profile obtained as per the method of the present disclosure. The present disclosure only provides a non-limiting list of such markers. Similarly, as per the method disclosed in the present disclosure, the increased expression of the biomarker of VSELs such as Oct4A, Stella, and Fragilis is indicative of an underlying medical condition or that of an inflammation present in the human subject. Therefore, it can be contemplated that the absence of any such increase is indicative of a healthy individual. The present disclosure only provides a non-limiting list of diseases that can be detected, however, depending on the type of markers used, any disease can be detected. Further, it is understood that once the entire sequence and transcriptomic profile is obtained from a simple blood sample, the information of the genetic profile can be used to provide complete information on the genetic, or transcriptomic level of a human subject.

An algorithm is defined as wherein the mutation, and expression data of very small embryonic-like stem cells will be cross-referenced with the scientific literature and human transcriptome/gene expression databases to identify a set of genes associated with a medical condition. The algorithm can connect transcriptome and whole-genome data to generate readings for tissue-level transcriptome data. Furthermore, based on the data, the organ parameters such as its functional activity, indicators of inflammation, oxidative stress, biological pathways, molecular mechanisms etc. would also be identified. Based on the identification of primary and secondary organs associated with the transcriptome and mutation data using the algorithm, delineating the susceptibility to a variety of medical conditions would also be possible. Further, the method of the present disclosure also enable testing for rare diseases such as and not limiting to spinal muscular dystrophy, Ehlers-Danlos syndrome, Proteus syndrome, sickle cell anemia, Hutchinson-Gilford progeria, etc. that are the end result of specific genetic mutations.

The method as per the present disclosure involves a process wherein very small embryonic-like stem cells are to be subjected to proteomics, metabolomics, methylation analysis, and the data acquired is connected through pathway analysis using various pathway databases to gene expression levels. The genetic analysis of VSELs along with pathway analysis leads to identification of disease treatment modalities (oncotherapy or disease specific interventions) for aiding clinicians and doctors. Further, the cDNAs obtained from VSELs are used to further detect the presence of a diseased condition and/or also to provide treatment modalities for treating the diseased condition. Furthermore, transcriptomic analysis of VSELS can be used to detect diseased condition and provide treatment modalities. As an alternative, exome analysis can also be performed on VSELs to detect diseased condition and provide treatment modalities for treating the diseased condition.

In an embodiment of the present disclosure, there is provided an in-vitro method for detecting a medical condition in a subject, said method comprising: (a) obtaining a blood sample and adding a salt solution to the blood sample; (b) layering the blood sample of step (a) over a neutral buffer and subjecting the blood sample to a density gradient centrifugation at a speed in the range of 200-900 g to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) subjecting the second pellet to a lysis for obtaining nucleic acid from the enriched very small embryonic like stem cells; (f) performing an assay with the nucleic acid of step (e) for analyzing expression level of Oct 4A in the very small embryonic like stem cells; and (g) comparing the expression level of Oct 4A in the very small embryonic like stem cells from the blood sample with an expression level of Oct 4A in a control sample for detecting an increase or decrease in the expression level, wherein an increase in the range of 1.1-3 folds in the expression level of Oct 4A in the very small embryonic like stem cells from the blood sample as compared to the expression level of Oct 4A in a control sample detects the presence of a medical condition in the subject. In one of the embodiments, subjecting the blood sample to a density gradient centrifugation is at a speed in the range of 200-500 g (preferably 200-300 g, most preferably 200 g) to obtain a first pellet comprising red blood cells (RBC). In another embodiment, centrifuging the RBC-lysed solution is at a speed in the range of 500-3000 g (preferably 700-2000 g, most preferably 1000 g) to obtain a second pellet comprising enriched very small embryonic like stem cells.

In an embodiment of the present disclosure, there is provided an in-vitro method for predicting onset of cancer or predicting the presence of tumor or cancer in a subject, said method comprising: (a) obtaining a blood sample and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) subjecting the second pellet to a lysis for obtaining nucleic acid from the enriched very small embryonic like stem cells; (f) performing an assay with the nucleic acid of step (e) for analyzing expression level of Oct 4A in the very small embryonic like stem cells; and (g) comparing the expression level of Oct 4A in the very small embryonic like stem cells from the blood sample with an expression level of Oct 4A in a control sample for detecting an increase or decrease in the expression level, wherein an increase in the range of 3-5 folds in the expression level of Oct 4A in the very small embryonic like stem cells from the blood sample as compared to the expression level of Oct 4A in the control sample predicts the onset of cancer in the subject, and wherein an increase of at least 5 folds in the expression level of Oct 4A in the very small embryonic like stem cells from the blood sample as compared to the expression level of Oct 4A in the control sample detects the presence of tumor or cancer in the subject.

In an embodiment of the present disclosure there is provided an in-vitro method for predicting onset of cancer or predicting the presence of tumor or cancer in a subject, wherein an increase in the expression level of Oct 4A in very small embryonic like stem cells in the blood sample as compared to the expression level of Oct 4A in the control sample in the range of 5-10 folds indicates stage-I of cancer, in the range of 10-15 folds indicates stage-II of cancer, in the range of 15-20 folds indicates stage-III of cancer, and in the range of 20-25 folds or more indicates stage-IV of cancer.

In an embodiment of the present disclosure, there is provided a method for detecting presence of a medical condition in a subject, said method comprising: (a) obtaining a blood sample from a subject and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) enumerating the number of very small embryonic like stem cells in the second pellet; and (f) comparing the number of very small embryonic like stem cells in the blood sample with the number of very small embryonic like stem cells in a control blood sample for detecting an increase or decrease in the number of very small embryonic like stem cells, wherein an increase in the number of very small embryonic like stem cells in the blood sample by at least two folds as compared to the number of very small embryonic like stem cells in a control blood sample detects the presence of a medical condition in the subject.

In an embodiment of the present disclosure, there is provided a method for detecting presence of tumor or cancer or onset of cancer in a subject, said method comprising: (a) obtaining a blood sample from a subject and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 200-4000 g to obtain a second pellet comprising enriched very small embryonic like stem cells; (e) enumerating the number of very small embryonic like stem cells in the second pellet; and (f) comparing the number of very small embryonic like stem cells in the blood sample with the number of very small embryonic like stem cells in a control blood sample for detecting an increase or decrease in the number of very small embryonic like stem cells, wherein an increase in the number of very small embryonic like stem cells in the blood sample in the range of 2-5 folds as compared to the number of very small embryonic like stem cells in a control blood sample detects the onset of cancer in the subject; and wherein an increase in the number of very small embryonic like stem cells in the blood sample by at least 5 folds as compared to the number of very small embryonic like stem cells in a control blood sample detects the presence of tumor or cancer in the subject.

In an embodiment of the present disclosure, there is provided an in-vitro method for detecting a positive response to anti-cancer therapy, said method comprising: (a) obtaining a blood sample-I before administration of an anti-cancer therapy; (b) enriching very small embryonic like stem cells from the blood sample-I by a process comprising: (bi) adding a salt solution to the blood sample I (bii) layering the blood sample-I of step (bi) over a neutral buffer and subjecting the blood sample to a density gradient centrifugation at a speed in the range of 200-900 g, to obtain a first pellet comprising red blood cells (RBC); (biii) lysing the RBC in the first pellet to obtain a RBC-lysed solution; (biv) centrifuging the RBC-lysed solution at a speed in the range of 400-4000 g to obtain a second pellet comprising enriched very small embryonic like stem cells from the blood sample-I; (c) obtaining a blood sample-II after at least 2 weeks of administration of the anti-cancer therapy; (d) enriching very small embryonic like stem cells from the blood sample-II by a process comprising: (di) adding a salt solution to the blood sample II, (dii) layering the blood sample-II of step (di) over a neutral buffer and subjecting the blood sample to a density gradient centrifugation at a speed in the range of 200-900 g, to obtain a first pellet comprising red blood cells (RBC); (diii) lysing the RBC in the first pellet to obtain a RBC-lysed solution; (div) centrifuging the RBC-lysed solution at a speed in the range of 400-4000 g to obtain a second pellet comprising enriched very small embryonic like stem cells from the blood sample-II; (e) obtaining nucleic acid-I from the enriched very small embryonic like stem cells from the blood sample-I; (f) obtaining nucleic acid-II from the enriched very small embryonic like stem cells from the blood sample-II; (g) independently performing an assay with the nucleic acid-I and the nucleic acid-II for analysing expression level of Oct 4A; and (h) comparing the expression levels of Oct 4A from the nucleic acid-II with the expression level of Oct 4A from the nucleic acid-I for detecting an increase or decrease in the expression level, wherein a decrease in the expression level of Oct 4A from the nucleic acid-II as compared to the expression level of Oct 4A from the nucleic acid-I detects a positive response to the anti-cancer therapy.

In an embodiment of the present disclosure, there is provided an in-vitro method for detecting cancer, said method comprising: (a) obtaining a blood sample and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) subjecting the second pellet to a lysis for obtaining nucleic acid from the enriched very small embryonic like stem cells; (f) performing an assay with the nucleic acid for analysing expression level of Oct 4A in very small embryonic like stem cells; (g) comparing the expression level of Oct 4A in very small embryonic like stem cells in the sample with an expression level of Oct 4A in very small embryonic like stem cells in a control sample for detecting an increase or decrease in the expression level, wherein an increase in the expression level of Oct 4A in very small embryonic like stem cells in the sample by at least 5 folds as compared to the expression level of Oct 4A in very small embryonic like stem cells in the control sample indicates presence of cancer; and (h) performing sequence-based assays on the nucleic acid and analyzing for mutation in at least one cancer-related marker, wherein presence of mutation in the at least one cancer-related marker indicates presence of a specific type of cancer based on the cancer-related marker analysed. In another embodiment, the cancer-related marker is selected from the group consisting of OLRI, CD68, MS R1, CXCL16, NCAN, TKTL1, ANO4, CHIT1, GPNMB, CCL18, TGFbeta1, FSP1, S100A6, SLC13A3, BGN, NCF2, 6Ckine, MMP-9, MMP-3, MMP-7, Integrin-04, Pleiotrophin, urokinase R, HLA-C, SLC9A3R1, NAT9, RAPTOR and SLC12A8, SPINK5, FcepsilonRI-beta, PHF11, IGFBP1, FACL4, ILIR, TGFbeta, CHRNA3/5, IREB2, HHIP, FAML3A, AGER, Troponin T&I, HSP60, BNP, GDF-15, MMP2, MMP3, MMP9, IL6, TNFalpha, CRP, SOX9, ACAN, COL2A1, DKK1, FRZB, RUNX2, COL10A1, IGH, IGHM, IGHG1, Sirtuins, ACE2, IFI27, IFIT1, IFITML, DPP4, KRAS, BRCA1 and 2, TP53, HLA-DQAI, HLA-DQB1, HLA-DRB1 (Type I), PPARG, KCNJ11, CDKAL1, CDKN2A-CDKN2B, IDE-KIF11-HHEX, IGF2BP2 and SLC30A8 (Type II), and combinations thereof. In yet another embodiment, the method further comprises co-relating a sequence profile with a reference sequence profile to identify the presence or absence of a mutation in the marker which is done by an algorithm.

In an embodiment of the present disclosure, there is provided a method for treating cancer, said method comprising: (a) obtaining a blood sample from a subject and adding a salt solution to the blood sample; (b) layering the blood sample of step (a) over a neutral buffer and subjecting the blood sample to a density gradient centrifugation at a speed in the range of 200-900 g, to obtain a first pellet comprising red blood cells (RBC); (c) lysing the RBC in the first pellet to obtain a RBC-lysed solution; (d) centrifuging the RBC-lysed solution at a speed in the range of 400-4000 g to obtain a second pellet comprising enriched very small embryonic like stem cells; (e) subjecting the second pellet to a lysis for obtaining nucleic acid from the enriched very small embryonic like stem cells; (f) performing an assay with the nucleic acid of step (e) for analysing expression level of Oct 4A in the very small embryonic like stem cells; (g) comparing the expression level of Oct 4A in very small embryonic like stem cells in the sample with an expression level of Oct 4A in a control sample for determining an increase or decrease in the expression level, wherein an increase in the expression level of Oct 4A in very small embryonic like stem cells in the sample by at least 5 folds as compared to the expression level of Oct 4A in the control sample detects cancer; and (h) administering anti-cancer therapy to the subject for treating cancer, wherein the administration of anti-cancer therapy decreases the expression level of Oct 4A in the subject.

In an embodiment of the present disclosure, there is provided a method for detecting the presence of a medical condition in a subject, said method comprising: (a) obtaining a blood sample from a subject and diluting the sample with a salt solution; (b) enumerating the number of very small embryonic like stem cells in the blood sample; and (c) comparing the number of very small embryonic like stem cells in the blood sample with the number of very small embryonic like stem cells in a control blood sample for detecting an increase or decrease in the number of very small embryonic like stem cells, wherein an increase in the number of very small embryonic like stem cells in the blood sample by at least 2 folds as compared to the number of very small embryonic like stem cells in a control blood sample detects the presence of a medical condition in the subject.

In an embodiment of the present disclosure, there is provided a method for predicting the onset of cancer or detecting presence of tumor or cancer in a subject, said method comprising: (a) obtaining a blood sample from a subject and diluting with a salt solution; (b) enumerating the number of very small embryonic like stem cells in the blood sample; and (c) comparing the number of very small embryonic like stem cells in the blood sample with the number of very small embryonic like stem cells in a control blood sample for detecting an increase or decrease in the number of very small embryonic like stem cells, wherein an increase in the number of very small embryonic like stem cells in the blood sample in the range of 2-5 folds as compared to the number of very small embryonic like stem cells in a control blood sample predicts the onset of cancer in the subject, and wherein an increase in the number of very small embryonic like stem cells in the blood sample by at least 5 folds as compared to the number of very small embryonic like stem cells in a control blood sample detects the presence of tumor or cancer in the subject.

In an embodiment of the present disclosure, there is provided a method for detecting the presence of a medical condition in a subject, said method comprising: (a) enumerating the number of very small embryonic like stem cells in vivo in blood of a subject; and (b) comparing the number of very small embryonic like stem cells in the blood of the subject with the number of very small embryonic like stem cells in a control blood sample for detecting an increase or decrease in the number of very small embryonic like stem cells, wherein an increase in the number of very small embryonic like stem cells in the blood of the subject by at least 2 folds as compared to the number of very small embryonic like stem cells in the control blood sample detects the presence of a medical condition in the subject.

In an embodiment of the present disclosure, there is provided a method for predicting the onset of cancer or presence of tumor or cancer in a subject, said method comprising: (a) enumerating the number of very small embryonic like stem cells in vivo in blood of a subject; and (c) comparing the number of very small embryonic like stem cells in the blood of the subject with the number of very small embryonic like stem cells in a control blood sample for detecting an increase or decrease in the number of very small embryonic like stem cells, wherein an increase in the number of very small embryonic like stem cells in the blood of the subject in the range of 2-5 folds as compared to the number of very small embryonic like stem cells in the control blood sample predicts the onset of cancer in the subject, and wherein an increase in the number of very small embryonic like stem cells in the blood of the subject by at least 5 folds as compared to the number of very small embryonic like stem cells in the control blood sample predicts the presence of tumor or cancer in the subject.

In an embodiment of the present disclosure, there is provided an in-vitro method as described herein, wherein the blood sample is peripheral blood sample.

In an embodiment of the present disclosure, there is provided an in-vitro method as described herein, wherein obtaining nucleic acid from the enriched very small embryonic like stem cells is by any one method selected from a group consisting of: (a) guanidinium thiocyanate-phenol-chloroform nucleic acid extraction; (b) cesium chloride gradient centrifugation method; (c) cetyltrimethylammonium bromide nucleic acid extraction; (d) alkaline extraction; (e) resin-based extraction; and (f) solid phase nucleic acid extraction. In another embodiment, the nucleic acid is RNA

In an embodiment of the present disclosure, there is provided an in-vitro method as described herein, wherein performing an assay with the nucleic acid for analysing the expression of Oct 4A is done by a technique selected from a group consisting of: quantitative PCR, flow cytometry, and Next Generation Sequencing (NGS).

In an embodiment of the present disclosure, there is provided a method for identifying a medical condition in a subject as described herein, wherein the medical condition identified is selected from the group consisting of multiple sclerosis, kidney disorders, skin disease, liver disease, lung disease, cardiovascular diseases, osteoarthritis, viral disease, cancer, and diabetes.

In an embodiment of the present disclosure, there is provided a method as described herein, wherein the very small embryonic-like stem cell have a size lesser than 7 microns in diameter. In another embodiment of the present disclosure, the very small embryonic-like stem cell has a size in the range of 1-7 microns in diameter. In an alternate embodiment of the present disclosure, the very small embryonic-like stem cell has a size in the range of 2-6 microns in diameter

In an embodiment of the present disclosure, there is provided a method as described herein, wherein lysing the RBC in the first pellet comprises treating the first pellet with a solution comprising ammonium chloride.

In an embodiment of the present disclosure, there is provided a method as described herein, wherein the enumerating is done by flow cytometry

In an embodiment of the present disclosure, there is provided a method as described herein, wherein the neutral buffer is Ficoll Hypaque solution.

In an embodiment of the present disclosure, there is provided a method as described herein, wherein the control sample is the expression level of a housekeeping gene from the subject. In another embodiment of the present disclosure, the housekeeping gene is 18s rRNA.

In an embodiment of the present disclosure, the VSELs isolated from the blood of a healthy individual is to be used for therapeutic applications. The VSELs are to be enriched in-vitro by promoting cell expansion and is to be edited using CRISPR-Cas9 technology for therapeutic applications. Alternatively, the VSELs are to be differentiated into tissue-specific cell types under suitable conditions and used for appropriate therapeutic application. As per another implementation, the VSELS are to be de-differentiated into induced pluripotent stem cells (iPSCs). The iPSCs can be further differentiated into tissue-specific cells which can be injected into the site of injury for therapeutic application. In an alternate implementation, there is provided a kit comprising reagents for enriching VSELs from a blood sample.

In an embodiment of the present disclosure, there is provided a method as described herein, wherein the control sample is obtained from a cancer-free subject.

In an embodiment of the present disclosure, there is provided a method as described herein, wherein the method is independent of invasive techniques.

In an embodiment of the present disclosure, there is provided a method as described herein, wherein the method is able to detect cancer without the intratumor heterogeneity.

In an embodiment of the present disclosure, there is provided a method for method for detecting the presence of a medical condition in a subject, wherein the method encompasses all the organs of a human subject in identifying a medical condition. In another embodiment, the method provides information equivalent to all organ biopsies.

In an embodiment of the present disclosure, there is provided a method as described herein, wherein an increase in the expression level of Oct 4A from very small embryonic-like stem cell from the sample as compared to the expression level of Oct 4A in the control sample differentiates malignant from benign conditions.

In an embodiment of the present disclosure, there is provided a method as described herein, wherein an increase in the expression level of Oct 4A from very small embryonic-like stem cell from the sample as compared to the expression level of Oct 4A in the control sample indicates mitochondrial alterations.

In an embodiment of the present disclosure, there is provided a method as described herein, wherein the method analyses any gene listed in the NCBI gene list database (https://www.ncbi.nlm.nih.gov/gene/) in VSELs, extracted from the blood, that when modulated as compared to control subjects, which is measured by expression analysis and mutation analysis of transcriptome and/or mutational analysis of exome and genome, indicates a medical condition with tissue-specific localization.

In an embodiment of the present disclosure, there is provided detection kit comprising, (a) primer set for analysing expression level of Oct4A in a mixture comprising very small embryonic-like stem cell; (b) reagents for performing quantitative PCR assay; (c) reagents for performing whole genome or exome or mitochondrial genome or transcriptome sequencing; and (d) at least one tissue-specific array for analysing a sequence profile.

Although the subject matter has been described with reference to specific implementations, this description is not meant to be construed in a limiting sense. Various modifications of the disclosed implementations, as well as alternate implementations of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with a working example, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Materials and Methods

### Clinical Study Design

The study as per the present disclosure was conducted after taking ethics approval from Ethics Committee of Maharashtra Technical Education Society at Sanjeevan Hospital, Pune, India and was registered with Clinical Trial Registry India (CTRI/2019/01/017166).

Initially 180 samples were collected along with patient history and all the related information. Oct-4A mRNA expression was studied in the VSELs enriched from the peripheral blood and helped arrive at a scale (HrC scale) wherein cancer stage was corelated to fold change in OCT-4A expression. Once the scale was obtained, its validation was done in a total of 1051 subjects, who were recruited from seven different sites across India for the study, out of which 500 were non-cancer and 500 were cancer patients (Table 1). The samples were blinded by National Facility for biopharmaceuticals. The patients with histologically or cytologically proven malignancy either solid tumors or haematological malignancy were included in the cancer group. Informed consent form was obtained from every subject. Circulating tumor cells (CTCs) were studied randomly in few cases on interest.

### Example 1

### Blood Sample Processing

Blood samples (approximately 10 ml of peripheral blood) were collected from the subjects and processed to enrich VSELs as described below. The blood sample was diluted with salt solution (1:1), preferably DPBS (composition of Dulbecco's phosphate buffered saline: NaCl. KCl, Na₂HPO4, KH₂PO4). Briefly, the samples were layered over Ficoll-Hypaque, i.e., the neutral buffer (ficoll: sample is 1:4) and subjected to density gradient centrifugation at 1200 rpm (200 g) for 15 min. Post centrifugation, a pellet (first pellet) was collected comprising RBCs and VSELs. The first pellet was subjected to lysis using ammonium chloride solution (as well known in the art) and then centrifuged at 3000 rpm (1000 g) to obtain a second pellet comprising VSELs.

### Example 2

RNA Isolation and cDNA Synthesis

Total RNA was extracted from the VSEL pellet (second pellet) using RNAplus (MP Biomedicals, Irvine, USA) according to manufacturer's instructions. After RNA extraction, first-strand cDNA was synthesized using the Revert Aid First strand cDNA synthesis kit (Thermo scientific, UK) according to the manufacturer's instructions. Briefly, 1 μg of total RNA was incubated with 5× Reaction Buffer and reverse transcriptase mix. The reaction was carried out in Applied Biosystems GeneAmp® thermal cycler 9700 (Applied Biosystems, USA) as per manufacturer's instructions.

### Example 3

qRT-PCR Studies

The expression level of Oct4A gene transcript was estimated by real-time PCR system-ABI 7500 (Applied Bio-systems, USA) using Thermo Scientific Maxima SYBR Green/ROX qPCR Master Mix kit (Thermo scientific, UK) and gene specific primer sequences, namely, Oct4A: Forward AGCCCTCATTTCACCAGGCC (SEQ ID NO: 1), and Reverse TGGGACTCCTCCGGGTTTTG (SEQ ID NO: 2). The 18s rRNA gene was used as housekeeping gene. The amplification conditions were: initial denaturation at 94° C. for 3 min followed by 45 cycles comprising of denaturation at 94° C. for 30 s, primer annealing at 62° C. for 30 s, and extension at 72° C. for 30 s followed by melt curve analysis step from 55° C. to 95° C. The fluorescence emitted was collected during the extension step of each cycle. The homogeneity of the PCR amplicons was verified by studying the melt curve. Ct values generated in each experiment using the 7500 Manager software (Applied Bio-systems, UK) were used to calculate the mRNA expression levels.

### Example 4

### Circulating Tumor Cells (CTCs)

CTCs were studied as described earlier (Diehl et al 2018). CTCs are found in patients with solid tumors and function as a seed for metastasis (Palmirotta et al 2018). They are considered as clinical biomarker and therapeutic target and are considered as a component of liquid biopsy. The peripheral blood was drawn into EDTA tubes. Within one hour, the tubes were subjected to centrifugation at 820 g for 10 min. Approximately 1-ml aliquots of the plasma was transferred to 1.5-ml tubes and centrifuged at 16,000 g for 10 min to pellet any remaining cellular debris. The supernatant was transferred to fresh tubes and stored at −80° C. Total genomic DNA was purified from 2 ml of the plasma aliquots using the QIAamp MinElute kit (Qiagen) according to the manufacturer's instructions. The amount of total DNA isolated from plasma was quantified with a modified version of a human LINE-1 quantitative real-time PCR assay, as described previously (Diehl et al 2008). The amount of total DNA isolated from plasma samples was quantified. Three primer sets were used to amplify differently sized regions within the most abundant consensus region of the human LINE-1 family (79 bp for: 5′-agggacatggatgaaattgg-3′ (SEQ ID NO: 3). 79 bp rev: 5′-tgagaatatgcggtgtttgg-3′ (SEQ ID NO: 4); 97 bp for: 5′-tggcacatatacaccatggaa-3′ (SEQ ID NO: 5), 97 bp rev: 5′-tgagaatgatggtttccaatttc-3′ (SEQ ID NO: 6); 127 bp for: 5′-acttggaaccaacccaaatg-3′ (SEQ ID NO: 7), 127 bp rev: 5′-tcatccatgtccctacaaagg-3′ (SEQ ID NO: 8). PCR was performed in a 25 μl reaction volume consisting of template DNA equal to 2 μl of plasma, 0.5 U of Taq DNA Polymerase, Ix PCR buffer, 6% (v/v) DMSO, 1 mM of each dNTP, 5 μl of SYBR Green and 0.2 μM of each primer. Amplification was carried out in Cycler using the following cycling conditions: 94° C. for 1 min; 2 cycles of 94° C. for 10 s, 67° C. for 15 s, 70° C. for 15 s; 2 cycles of 94° C. for 10 s, 64° C. for 15 s, 70° C. for 15 s, 2 cycles of 94° C. for 10 s, 61° C. for 15 s, 70° C. for 15 s; 35 cycles of 94° C. for 10 s, 59° C. for 15 s, 70° C. for 15 s. The results with CTCs are summarized in Table 2.

### Results

Initially a HrC scale was developed (as explained hereinabove) based on Oct-4A expression in 180 samples. The Oct4A expression in peripheral blood was correlated with the medical history (PET scan and biopsy reports). It was observed that Oct4A was manifold upregulated in peripheral blood of cancer patients compared to non-cancer subjects. Within cancer patients, the expression of OCT4A was highest for stage 4 cancer and lowest for stage 1. On the basis of fold increase, an HrC scale was developed using which non-cancer and cancer subjects can be segregated. The HrC scale/value as per the present disclosure was designed in a manner that the HrC value is double the fold change in the expression of Oct 4A analyzed from the blood samples of the test subject as compared to a housekeeping gene or Oct 4A analyzed from the blood sample of a healthy subject. For clarity purposes, if the fold change in the expression of Oct 4A is X, then the HrC value would be 2X. Also, the stage of the cancer was deciphered. The non-cancer patients and those with increased inflammation that could lead to cancer initiation (on correlating with patient history) in future also revealed specific range of values. The subjects were identified and distributed on the basis of their HrC score as non-cancer, inflammation, high risk, stage I cancer, stage II cancer, stage III cancer and stage IV cancer (FIG. 1).

Between January to May 2019, total of 1051 patients were screened and recruited for the study. 51 subjects out of 1051 were excluded because of screen failures. Therefore, a total of 1000 patient samples were analyzed. There were 534 males and 466 females. The median patient age was 63.0 years for the complete dataset. The mean weight was 69.3 kg and mean height was 161.38. Table 1 summarizes patient demographics for the complete dataset.

TABLE 1
Demographics of all the subjects included in the study

| | |
|---|---|
| Patient population | Number of participants |

Gender

| | |
|---|---|
| Male | 534 |
| Female | 466 |

Age (years)

| | |
|---|---|
| Mean | 61.3 |

Weight (kg)

| | |
|---|---|
| Mean | 69.3 |

Height (cm)

| | |
|---|---|
| Mean | 161.38 |

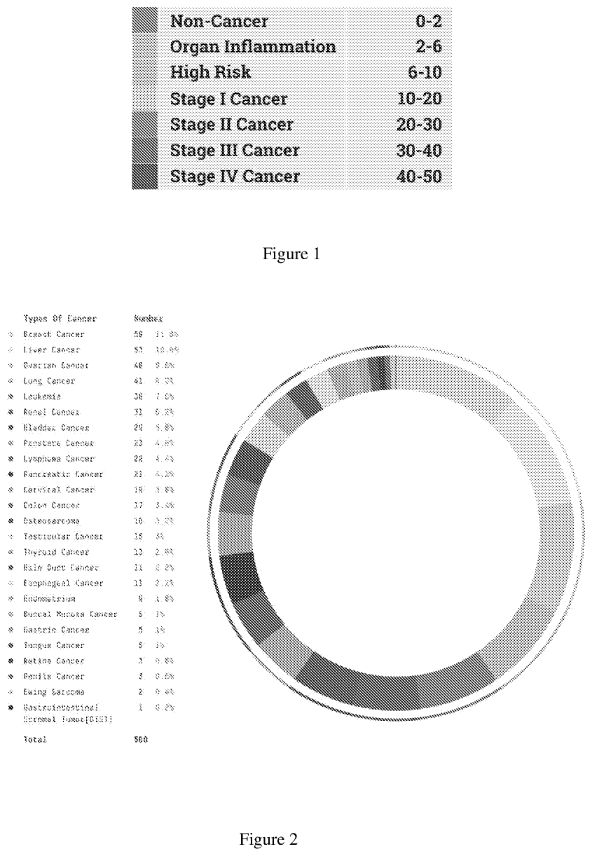

Out of 500 cancer patients, 431 patients were on treatment (Rₓ), 48 were not subjected to any treatment after diagnosis of cancer (Rₓ Naïve) and 21 patients had undergone surgical intervention for cancer treatment (R₀). Patients with 25 different types of cancers were included in the study as shown in FIG. 2.

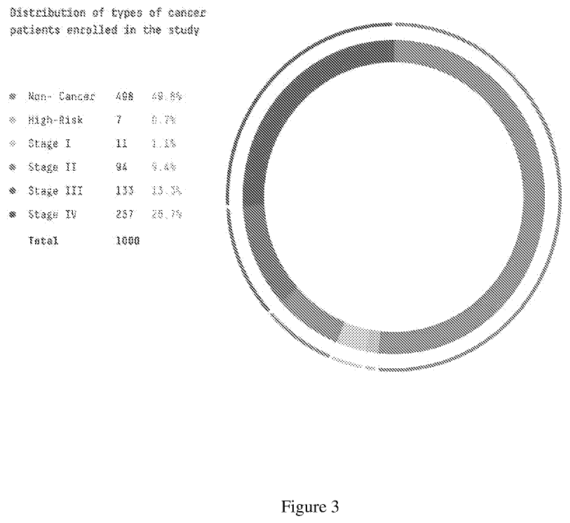

Out of 1000 samples analyzed for HrC, 498 samples were non-cancerous, 7 were assessed to be in high-risk stage, 11 were in stage I cancer, 94 were in stage II cancer, 133 were in stage III cancer, and 257 were in stage IV cancer (FIG. 3).

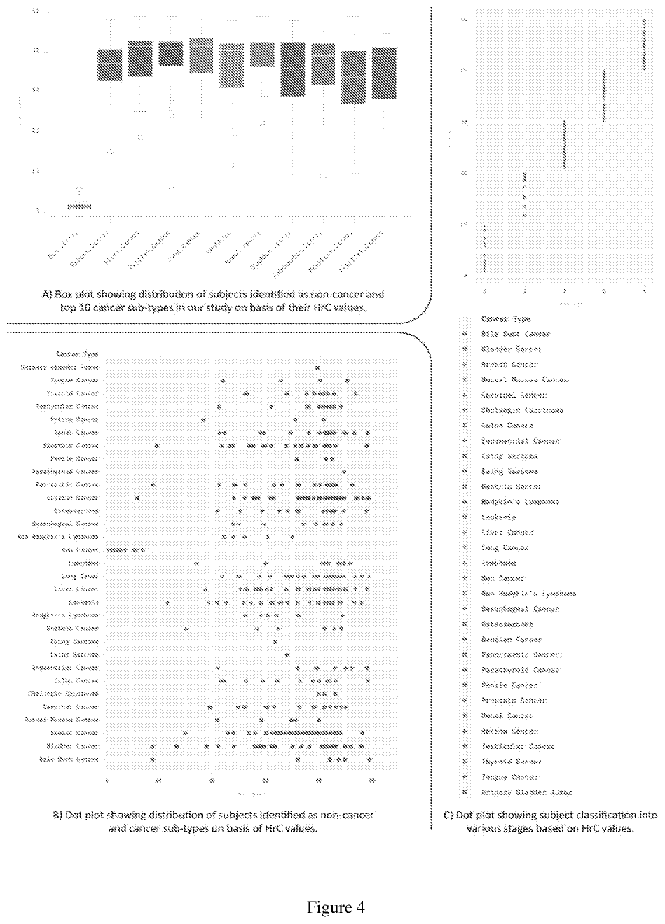

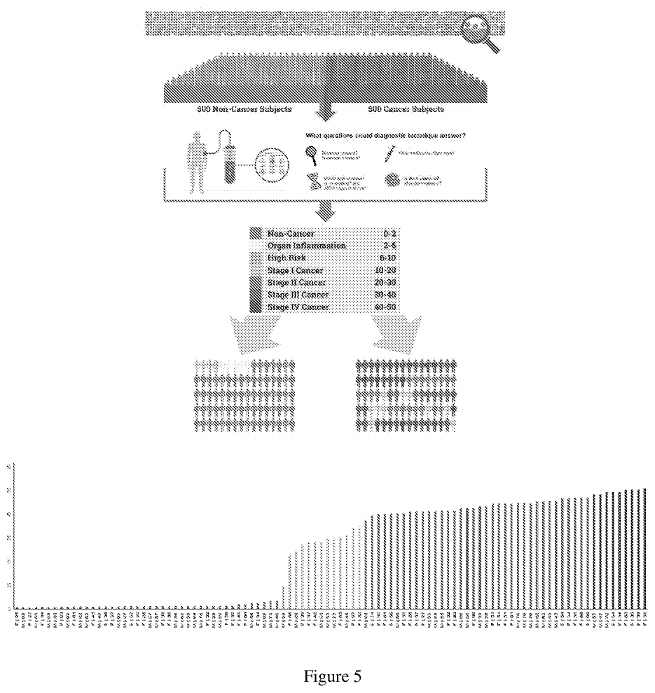

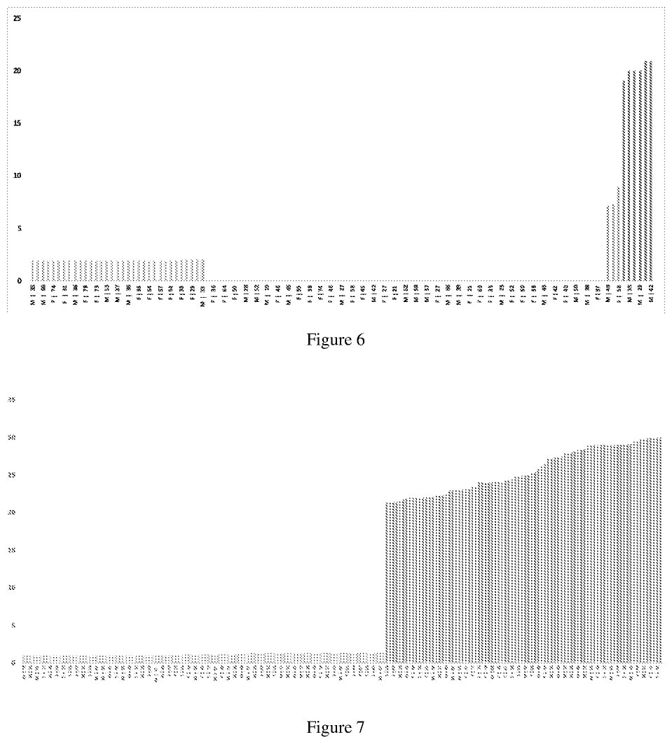

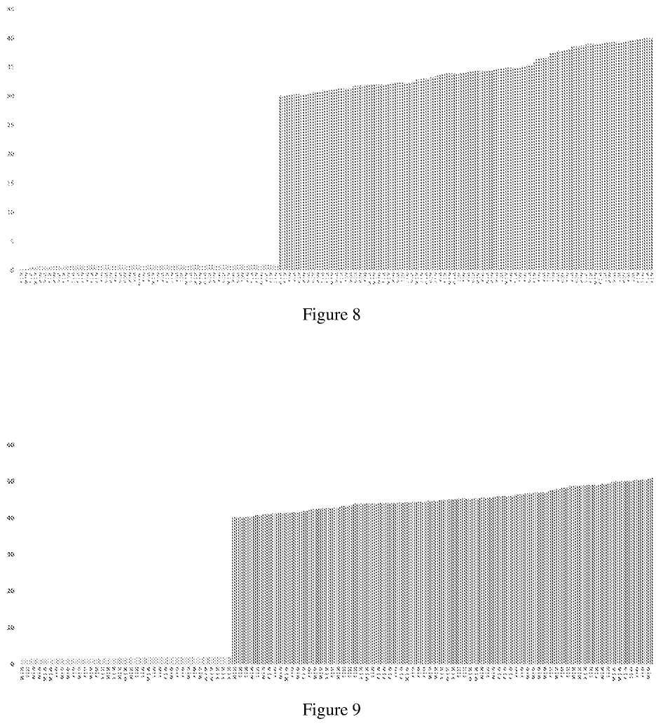

HrC levels were able to detect presence of several types of solid and liquid cancers. FIGS. 4-9 provides details of the results in all the 1000 study subjects. As explained in the preceding paragraphs, the HrC scale/value as per the present disclosure was designed in a manner that the HrC value is double the fold change in the expression of Oct 4A analyzed from the blood samples of the test subject as compared to a housekeeping gene or Oct 4A analyzed from the blood sample of a healthy subject. For clarity purposes, if the fold change in the expression of Oct 4A is X, then the HrC value would be 2X. As evident from FIGS. 4-9 there was no ambiguity in the HrC values. Table 2 provides details of 10 cases where novel results were obtained using HrC as a tool to monitor cancer state of patients.

TABLE 2
Details of 10 cases where the results were obtained using
HrC as a tool to monitor cancer state of patients.

| | HrC levels (as per the | |
|---|---|---|
| Patient details | present disclosure) | Remarks |
| 68 years old female with | HrC analysis revealed a score of 26.82 | Several researchers have |
| colorectal cancer. patient | indicating stage II cancer whereas | reported non-reliability |
| was subjected to HrC and | ctDNA (circulating tumor DNA) were | of ctDNA for accurate |
| CTCs analysis | not detected in the blood | diagnosis of cancer |
| | | (Shen et al 2018) |
| 55-years-old, female was | On the day of surgery, HrC value was | The HrC test |
| recruited in cancer group | 32.11. Four weeks post removal of | successfully aided |
| with stage III cervical | the tumor surgically, HrC value fell to | oncologists to monitor |
| adenocarcinoma | 9.14 which suggested that the patient | the disease progression |
| | still fell in high risk category and | and risk of relapse. |
| | required adjuvant chemotherapy. She | |
| | underwent four cycles of adjuvant | |
| | chemotherapy (Day 28 to Day 160) | |
| | and PET scan performed at the end | |
| | showed absence of lesion. The patient | |
| | was declared cancer- free, and the | |
| | HrC value showed a reading of 1.9 | |
| | (Day 167) in alignment with PET scan | |
| 74 years old male patient | HrC test was performed one day prior | HrC test could serve as a |
| was enrolled with Stage 4 | to neo-adjuvant chemotherapy and | reliable marker for |
| liver cancer | was 49.43 (indicating stage 4 cancer | oncologists to interpret |
| | patient). After three cycles of neo- | disease progression and |
| | adjuvant chemotherapy, HrC value | effectiveness of the |
| | dropped to 42.01. | treatment |
| 68-years-old, 76- Kg | HrC values were 9.78 which | The subject was |
| male was recruited in | suggested that he was a high risk (pre- | experiencing generalized |
| non-cancer group. | cancer individual). Based on HrC | body weakness, acidity, |
| | values, he was investigated in details | abdominal pain and a |
| | and enlargement of prostate was | weight loss. |
| | detected with PSA levels 182 ng/ml | Patient management was |
| | and blood sugar levels as 210 mg/dL | carried out efficiently |
| | and 170 mg/dL fasting and post | based on HrC test results |
| | prandial respectively. All the other | |
| | blood test including complete blood | |
| | count, blood urea nitrogen test, serum | |
| | uric acid, liver function test and lipid | |
| | test were normal. PET scan showed | |
| | no signs of lesion across the body. | |
| | After obtaining patient consent, | |
| | radical prostatectomy was performed. | |
| | On the day of surgery (9 weeks post | |
| | first HrC analysis), HrC value | |
| | increased to 10.89 from 9.78. Four | |
| | weeks post prostatectomy, HrC value | |
| | was reduced to 2.1 which indicated | |
| | slight organ inflammation. | |
| 47-year-old stage 3 | HrC value was 36.18 which classified | Core biopsy of the mass |
| breast cancer patient with | her as Stage III. Surprisingly, CTCs | showed metastatic breast |
| a 7.4 cm left breast mass. | were not detected in blood. | cancer, estrogen receptor |
| | | (ER) 95% positive, |
| | | progesterone receptor |
| | | 85% positive and HER2 |
| | | negative. ASCO tumor |
| | | marker guidelines (2007) |
| | | suggest that |
| | | measurement of CTCs |
| | | should not be used for |
| | | diagnosis or treatment |
| | | modifications. |
| 65-year-old female with | HrC value was 41.28 (Day 0) and | The doctors were unable |
| a tumor above ovary | cancer antigen 125 (CA-125) was | to detect primary site of |
| | 198.8 along with pain in abdomen | cancer and it was |
| | (Day 3). Patient underwent surgery | impacting the course of |
| | (Day 7) for the removal of both | treatment (Day 30). |
| | ovaries, uterus, and fallopian tube. | HrC test was able to |
| | Immuno-histochemistry analysis (Day | accurately detect the |
| | 10) of the removed tissue suggested | primary site of cancer as |
| | primary site of cancer be the stomach | the appendix by |
| | since the tissue was positive for CK- | analyzing the expression |
| | 20 & CDX2/SATB2. | profile of Day 44. |
| 49-year-old male non- | The subject was recruited in Non- | HrC test was able to |
| cancer subject | cancer group, upon analysis it was | screen subjects for the |
| | found that HrC score was 7.20 | development of cancer. |
| | indicating “high risk cancer” category. | |
| | In-depth analysis further revealed that | |
| | the subject was at the risk of | |
| | developing oral cancer. The patient | |
| | was an active user of pan masala and | |
| | gutkha and regular smoker | |
| 78-year-old cancer | The subject was recruited in cancer | HrC test was used to |
| patient with carcinoma of | group and HrC analysis showed value | assess the efficacy of the |
| lung and parenchymal | of 46.34 (indicating stage IV cancer) | chemotherapy treatment. |
| non- cerebral metastasis | (Day 0). Post two cycles of | |
| | chemotherapy (Day 36) diagnostic test | |
| | was conducted again to assess the | |
| | efficacy of the treatment. The HrC | |
| | test at Day 36 showed value of 42.38, | |
| | clearly indicating improvement in the | |
| | cancer. | |
| 52-year-old male non- | The subject was recruited in Non- | HrC test was able to |
| cancer subject | cancer group, upon analysis it was | detect high risk category |
| | found that HrC score was 7.86 | and the organ which was |
| | indicating “high risk cancer” category. | at the risk of developing |
| | In-depth analysis further revealed that | cancer. |
| | the subject was at the risk of | |
| | developing thyroid cancer. Upon | |
| | further consultation with Oncologist, | |
| | the subject underwent biopsy which | |
| | revealed benign hemorrhagic nodule | |
| | with degenerative changes and had | |
| | extremely high levels (224 pg/ml) of | |
| | calcitonin. The subject underwent | |
| | total thyroidectomy | |
| 68-year-old male patient | HrC value was 40.15 indicative of 4th | HrC test was able to |
| with liver cancer | stage liver cancer. A detailed | accurately detect stage |
| | mutational analysis revealed lymph | of cancer and primary |
| | node metastasis as per TNM | site of cancer by |
| | classification. Further mutation | analyzing the mutation |
| | analysis revealed primary and | and expression profile |
| | secondary organs as liver and lung | |
| | with osseous metastasis. | |
| | Cholangiocarcinoma was identified | |
| | as specific type of cancer and further | |
| | sub- localization was identified using | |
| | pathway analysis (data not shown). | |

### Example 5

Analyzing the Number of VSELs in the Blood of a Subject and its Correlation with a Medical Condition or Cancer in the Subject

The VSELs count per unit of blood can be measured to not only distinguish between people with cancer, imminent cancer and non-cancer but can also distinguish between stages of cancer. Invasive in vitro imaging of VSELs is done by routine colorimetric staining using nuclear staining approaches such as hematoxylin, Hoechst 33342 dye etc. once the cells are isolated from a unit of blood. Non-invasive optical microscopy, on the other hand, is a recently developed in vivo technique that takes advantage of confocal microscopy principles for imaging large cross-sectional areas of blood vessels with sub-micron resolution (thus, identifying, cells of interest in size range of 2-6 gm indicative of VSELs) without staining. One such example is through methods pertaining to electric or ultrasound waves can be utilized. The principle behind the technique is different light scattering coefficients of cellular and subcellular structures when incident on a particular blood vessel detected at a measured depth below the tissue surface. In another implementation, fluorescent-based techniques and image capturing of stained cells in blood flow can also be used, though this process may modify the cells and/or result in toxicity.

A novel approach has been developed in the present disclosure to quantify the number of Oct4+ positive cells in the pellet as shown below.
1. 3 ml of peripheral blood was collected from normal subjects and a cancer individual.
2. Blood was subjected to Ficoll-Hipaque density centrifugation at 200 g-900 g for 30 minutes.
3. Post RBC lysis of the resultant RBC pellet (first pellet), the supernatant was spun at 400-4000 g to obtain a second pellet comprised of distinct cell populations.
   To test the enrichment procedure, the second pellet was tested for Lin-CD45-Oct4+ populations via flow cytometry. Briefly, the second pellet was treated with following antibodies: —Lineage cocktail, anti-CD45 followed by cell fixation and permeabilization and later for anti-Oct4. Our preliminary analysis quantified 535 Lin-CD45-Oct4+ cells/ml in the second pellet for the non-cancer subject and 793 Lin-CD45-Oct4+ cells/ml in the second pellet for the cancer subject, thus, implying, a >1.45-fold increase in VSEL numbers in second pellet of a cancer patient as compared to control sample (Table 3). Thus, enumeration of Oct4 positive cells via flow cytometry indicates distinguishing a medical condition, particularly cancer, in the human subject.

TABLE 3
Oct4 positive cell populations in second pellet
for non-cancer vs. cancer subjects.

| | Cell | | % | Number | VSELs |
|---|---|---|---|---|---|
| OCT-4A | Type | Name | VSELs | of Events | per ml |
| COMPENSATED | VSELs | Non-Cancer | 0.42 | 1283 | 535 |
| COMPENSATED | VSELs | Cancer | 17.5 | 1890 | 793 |

*CD45 cells express Oct4 in nuclei that is Oct4a whereas CD45⁺ cells express Oct4 in cytoplasm i.e. Oct4b.

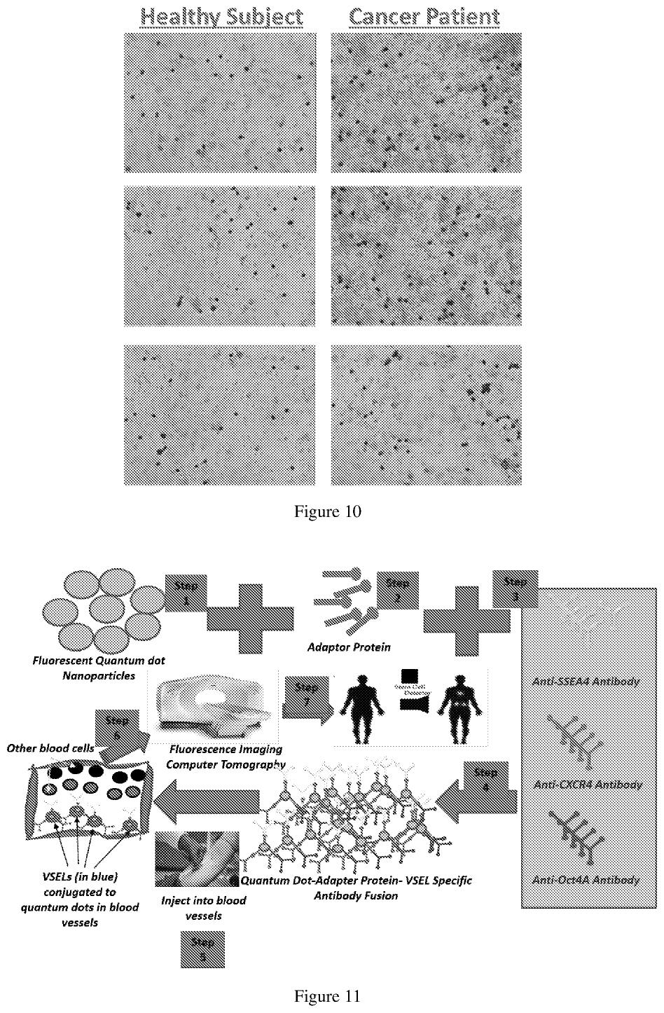

FIG. 10 depicts the comparison of the number of VSELs present in the blood of a cancer patient versus a healthy individual. The analysis was performed by isolating VSELs from peripheral blood of a fourth stage 65-year old female patient with Chronic Myeloid Leukemia (blood cancer), preparing smears, fixing in 4% paraformaldehyde, staining with Hematoxylin/Eosin and imaging using a microscope. Referring to FIG. 10, the left panel represents the blood sample from a healthy subject, and the right panel represents the blood sample from a cancer subject. As per the analysis of the image, the approximate number of VSELs in the top, middle, and bottom image of the left panel are 25, 22, and 22. On the other hand, the approximate number of VSELs in the top, middle, and bottom image of the right panel are 53, 55, and 52. Therefore, FIG. 10 clearly demonstrates that the increase in number of VSELs can be correlated with the presence of cancer.

### Example 6

### In-Vivo Detection of Medical Condition and Cancer

As per one implementation of the present disclosure, the quantity of VSELs in the blood can also be analyzed in-vivo. FIG. 11 depicts one of the many modalities which can be used to analyze the number of VSELs in the blood by in-vivo methodology. It is envisaged to develop a Bio-GPS system for cancer detection using fluorescent quantum dot nanoparticles (step 1), that when fused with intermediate adapter proteins (step 2) and VSEL-specific antibodies (step 3) results in quantum dot-adapter protein-VSEL specific antibody fusion molecules (step 4). This solution when injected into blood stream (step 5) results in tagging specifically of VSELs by quantum dots and selective fluorescence emission that can be captured via fluorescent imaging computer tomography (steps 6 and 7). Thus, in vivo VSEL image analysis can lead to contrast agent injection-mediated identification of VSEL count in normal vs. cancer patients.

The results of the study suggest that it is possible to predict, screen and diagnose cancer from a blood test. The results confirm the potential of HrC test (method as per the present disclosure) for reliable blood-based diagnosis of cancer. The specificity of HrC test was >99% with no false positives or false negatives. HrC test adopts a machine learning based algorithm for multi-analyte data to enable the cancer to be specifically identified. Data on ten interesting cases is provided where HrC analysis helped the clinician (Table 2).

### Example 7

Example of cDNA analysis of VSELs In a whole transcriptomic analysis, RNA fragments are converted to cDNA libraries for gene expression and mutational analysis. Thus, a transcriptomic analysis of VSELs from a fourth stage liver cancer patient was conducted and mutations were found in the genes as listed in Table 4 corresponding to various organ metastasis of cancer. As shown herewith, the highest number of gene mutations were obtained for bone lesions, though only 2 of those genes were non-intronic. On the other hand, liver also showed mutations in 2 non-intronic genes out of 3 while lung showed one 5UTR gene mutation as per COSMIC, ICGC databases. Hence, it can be contemplated that the cDNA information of the VSELs enriched from the peripheral blood of a subject can provide information to specifically identify the medical condition. For example, even in cases where the origin of cancer is not identified using the conventional methodologies, the cDNA obtained from VSELs enriched from the peripheral blood can provide information to this effect.

TABLE 4
Mutation profile analysis of 9 genes (obtained from the VSELs enriched
from peripheral blood of human subjects as per the present disclosure).

| | VAR | cDNA | AA | |
|---|---|---|---|---|
| Gene | CLASS | Change | change | Localization |
| GRIN2C | INFRAME- | c.3145_3146insCCCCGGAGC | p.Glu1048_Leu1049insProProGlu | Liver |
| | INS | | | |
| PRICICLE4 | INFRAME- | c.863_864insTCT | p.Leu288dup | Liver |
| | INS | | | |
| UNC50 | INTRONIC | c.−4-46T > A | NA | Liver |
| NR1I2 | 5UTR | c.−20dup | NA | Lung |
| PRKDC | FRAMESHIFT- | c.3729dup | p.Arg1244ProfsTer41 | Bone |
| | INS -S | | | |
| | S-PRX | | | |
| CSMD3 | INTRONIC | c.5605 + 51del | NA | Bone |
| CYFIP2 | INTRONIC | c.208-1815dup | NA | Bone |
| EIF1B | INTRONIC | c.298-33_298-32del | NA | Bone |
| ZCHHC6 | INFRAME- | c.2811_2813del | p.Lys937del | Bone |
| | DEL | | | |

### Example 8

### Enumeration of VSELs in Whole Blood

As per one implementation of the present disclosure, the quantity of VSELs can be enumerated in whole blood without the steps of lysis and washing. The steps for this process are provided hereinbelow:
1. 3 ml whole blood was collected from a normal person. From the 3 ml blood, I ml was taken and further diluted to 4 ml with 1× focusing fluid.
2. From the 4 ml diluted blood of step (1), 1 ml was taken and made up to 4 ml with ix focusing fluid.
3. The diluted blood of step (2) can directly be used to run unstained sample and to set the scatter of FSC and SSC plot by adjusting voltages.
No lyse no wash protocol is required to be further used for the enumeration of VSELs in the stained sample as per this implementation.

### SUMMARY

The present disclosure discloses a simple method (HrC test) for assessing the molecular profile of cancer (range 0-60) from the blood of a subject. The different range of scores was correlated to different stages of cancer using a third-degree polynomial equation comprising Oct4A gene expression levels and provides information for all types of cancers including whether (i) cancer is present (ii) cancer is imminent (iii) different stage of cancer and (iv) effect of oncotherapy. Further, the method disclosed by the present disclosure also tells whether the subject from which the sample (blood) is analyzed has any other medical condition apart from cancer. The HrC scale links VSEL Oct4A expression with a medical condition based on scoring of 0-2 which is indicative of absence of cancer/inflammation, and 2-6 which relates to presence of inflammatory status indicative of medical conditions such as diabetes, tuberculosis, Alzheimer's disease, dementia, cardiovascular disease, arthritis, etc. The non-cancer patients and those with increased inflammation which could lead to cancer initiation (on correlating with patient history) in future could also be classified based on HrC data. The results of the study suggest that it is possible to predict, screen and diagnose cancer from a blood test. The specificity of HrC test was >99% with no false positives or false negatives. HrC adopts a machine learning based algorithm. Cancer is a fatal, debilitating disease that accounted for >9 million deaths worldwide in 2018 (Bray et al 2018). The disease etiology is characterized by genetic alterations (Chakravarthi et al 2016) and metabolic changes (Hammoudi et al 2011) that transcend into uncontrollable, abnormal cellular growth, proliferation and metastatic progression (Riggi et al 2018). Late-stage cancers often lack an effective treatment option (Chakraborty and Rahman 2012). Currently, the need of the hour is to detect the disease as early as possible, since early-stage detection can result in aiding clinicians in identifying suitable interventions to prevent the onset or further progression of the disease, reduce treatment cost, improve patient outcome (disease-free and progression free survival, time in remission, delay relapse) on a case-by-case basis (Schiffman et al 2015). Nearly 70% of all cancers can be prevented if risk is detected at an early stage, thus, emphasizing need for better point-of-care diagnostics (Gandhi et al 2017). Average five-year survival rate at early stage is 75% whereas average five-year survival rate at late stage is merely 16% (Eskiizmir et al 2017). Current diagnostic methods include PET CT scan, MRI and the gold standard of all methods, the tissue biopsy (Cowling and Loshak 2019). Biopsy is expensive, invasive or painful, causing discomfort and the surgical procedures warrant with undue, resultant side-effects (Do et al 2019). Furthermore, due to inconspicuous anatomical locations, some tumor specimens are difficult to isolate making them inaccessible (Do et al 2019). Also, tissue biopsies might not give accurate information due to tumor heterogeneity in gene expression and mutations. Tissue biopsies may augment risk of metastatic lesions and safety is also a concern, for e.g. related to sampling of angiogenic tumor microenvironments (Do et al 2019). Similarly, imaging methods do not, at times, detect the cancer source, i.e., cancer of unknown primary (CUP) origin (Varadhachary 2007) is relatively frequent leading to inaccurate diagnosis affecting interventional therapies. Colonoscopy, prostate specific antigen, mammography and cervical cytology are limited number of existing screening test for a few number of cancer types (Ilic et al 2018); although their efficacy is questioned (Ilic et al 2018) and several patients do not follow medical guidelines for screening (Hie et al 2018). Majority of cancer types lack an effective non-invasive early screening option (Curry et al 2003).

The HrC scale was developed and tested on multiple cancer types on the basis of a pilot clinical study conducted with subjects registered with CTRI bearing number CTRI/2018/07/015116. This clinical study was performed to assess the Oct4A fold change expression values of cancer and noncancer subjects. The Oct4A expression of the subjects was correlated with their medical history (PET scan and biopsy reports) and it was observed that Oct4A was manifold upregulated in cancerous blood sample as compared to non-cancer subject. Within cancer patients the expression of OCT4A was highest for stage 4 cancer and lowest for Stage 1. Furthermore, in cancer subjects, stages of cancer were accurately identified on the basis of HrC scale.

The present disclosure discloses a method (HrC test) which involves isolating VSELs from blood and utilizing its associated pluripotency marker Oct4A with path-breaking implications as a diagnostic and prognostic tool with significant advantages over tumor cell-mediated cancer detection systems.

In case of imminent cancer detection, the method can lead to preventative strategies while HrC scale testing after oncotherapy can help determine disease survival rate, effect of treatment and probability of recurrence. Thus, Oct4A from VSELs, an oncogene, is described as the first pluripotent marker that can detect cancer and its stages with 100% sensitivity and specificity as per a trial of 500 non-cancer and 500 cancer patients. Mechanistically, this is primarily due to its constitutive activation in VSELs, defining its pluripotency, and hence the clinical manifestations of a) VSELs initiating cancer endogenously, b) VSELs transformation to cancer stem cells by yet unknown mechanisms, c) cancer stem cells as major drivers of malignancy, as well as invasiveness, migration and motility, d) detection of enriched VSELs in blood and Oct4A overexpression as an exclusive marker of primitive and malignant cell phenotype.

### Advantages of the Present Disclosure

The method as disclosed in the present disclosure, is a simple blood test and does not involve any invasive techniques. The method provides data equivalent to the information obtained through traditional biopsies, but without the invasive part. Also, a biopsy can only be performed if there is a cue about the tissue that could be damaged or is responsible for an underlying condition. In many cases, the human body might not give the early signals relating to an underlying medical condition because of which by the time the condition arises, the patient could be left with very less time at hand. In essence, the disclosure herein was able to establish the effective diagnostic scope of this non-invasive process to not only prognose and detect cancer earlier than current known technologies but also have the widest scope to detect significant variety of cancers (solid tumors, hematologic malignancies, and sarcomas) with a single marker. The ability of this method was also identified to provide mutational and expressions transcriptome data, analytical depth and pathways informational data to a level that is currently possible only through invasive biopsies and that too of multiple organs. In case of the presence of early signs of inflammation or a medical condition, the sequencing of the transcriptome, genome, mitochondrial genome, or exome, obtained from the VSELs can clearly pin-point the medical condition of the subject. Additionally, the sequencing data can also be used to accurately pin-point the type of cancer that is present in the subject.

Further, the present disclosure also includes the scope of a transcriptome gene bank. The transcriptome gene bank is a repository to store genetic material outside the organism in an in vitro setting for subsequent analysis at a later stage to assess health conditions. As a result, storage of RNA samples (−80° C. or even under liquid nitrogen), that are indicative of mutational and expression profiles of healthy as well as diseased individuals, can provide, at any time point, a dynamic analysis of the genetic alterations. Thus, RNA storage of individuals is of critical importance to detect diseased conditions temporally. VSELs can be readily obtained from the blood samples in a painless, fast, low-cost and non-invasive way and are also indicative of the dynamic tissue-specific gene expression profiles indicative of whole organ biopsy. Thus, the RNA bank storing genetic material of VSELs from a subject's blood sample can potentially provide rich data about the health condition of the individual from a whole body/organ perspective at any stage of the patient's lifetime. This data can be cross-referenced with other commercially available pathology tests to aid the clinicians and doctors in disease diagnosis and possibly suggesting treatment modalities.

### REFERENCE

Asai, N. et al. (2013) ‘Is emphysema a risk factor for pneumothorax in CT-guided lung biopsy?’, SpringerPlus, pp. 1-6. doi: 10.1186/2193-1801-2-196.
Bedard, P. L. et al. (2013) ‘Tumour heterogeneity in the clinic’, Nature, pp. 355-364. doi: 10.1038/nature12627.
Bowcock, A. M. and Cookson, W. O. C. M. (2004) ‘The genetics of psoriasis, psoriatic arthritis and atopic dermatitis’, Human Molecular Genetics. doi: 10.1093/hmg/ddh094.
Bray, F. et al. (2018) ‘Global cancer statistics 2018: GLOBOCAN estimates of incidence and mortality worldwide for 36 cancers in 185 countries’, CA: A Cancer Journal for Clinicians, 68(6), pp. 394-424. doi: 10.3322/caac.21492.
Chakraborty, S. and Rahman, T. (2012) ‘The difficulties in cancer treatment.’, Ecancermedicalscience, 6, p. ed16. doi: 10.3332/ecancer.2012.ed16.
Chakravarthi, B. V. S. K., Nepal, S. and Varambally, S. (2016) ‘Genomic and Epigenomic Alterations in Cancer’, American Journal of Pathology, pp. 1724-1735. doi: 10.1016/j.ajpath.2016.02.023.
Chaudhary, S. and Mittra, I. (2019) ‘Cell-free chromatin: A newly described mediator of systemic inflammation’, Journal of Biosciences. doi: 10.1007/s12038-019-9849-7.
Cleary, S. P. et al. (2013) ‘Identification of driver genes in hepatocellular carcinoma by exome sequencing’, Hepatology, 58(5), pp. 1693-1702. doi: 10.1002/hep.26540.
Cowling, T. and Loshak, H. (2016) An Overview of Liquid Biopsy for Screening and Early Detection of Cancer, CADTH Issues in Emerging Health Technologies. Available at:
http://www.ncbi.nlm.nih.gov/pubmed/32239883.
Dhingra, R. and Vasan, R. S. (2017) ‘Biomarkers in cardiovascular disease: Statistical assessment and section on key novel heart failure biomarkers’, Trends in Cardiovascular Medicine, pp. 123-133. doi: 10.1016/j.tcm.2016.07.005.
Diehl, F. et al. (2008) ‘Circulating mutant DNA to assess tumor dynamics’, Nature Medicine, 14(9), pp. 985-990. doi: 10.1038/nm.1789.
Ene, R. et al. (2015) ‘Synovial inflammation in patients with different stages of knee osteoarthritis’, Romanian Journal of Morphology and Embryology, 56(1), pp. 169-173.
Eskiizmir, G., Ermertcan, A. T. and Yapici, K. (2017) ‘Nanomaterials: Promising structures for the management of oral cancer’, in Nanostructures for Oral Medicine, pp. 511-544. doi: 10.1016/B978-O-323-47720-8.00018-3.
Francis, R. and Lewis, C. (2018) ‘Myocardial biopsy: Techniques and indications’, Heart, 104(11), pp. 950-958. doi: 10.1136/heartjn1-2017-311382.
Gandhi, A. K. et al. (2017) ‘Burden of preventable cancers in India: Time to strike the cancer epidemic’, Journal of the Egyptian National Cancer Institute, pp. 11-18. doi: 10.1016/j.jnci.2016.08.002.
Gerlinger, M. et al. (2012) ‘Intratumor Heterogeneity and Branched Evolution Revealed by Multiregion Sequencing’, New England Journal of Medicine, 366(10), pp. 883-892. doi: 10.1056/nejmoa1113205.
Hammoudi, N. et al. (2011) ‘Metabolic alterations in cancer cells and therapeutic implications’, Chinese Journal of Cancer, pp. 508-525. doi: 10.5732/cjc.011.10267.
Hannivoort, R. A., Hernandez-Gea, V. and Friedman, S. L. (2012) ‘Genomics and proteomics in liver fibrosis and cirrhosis’, Fibrogenesis and Tissue Repair. doi: 10.1186/1755-1536-5-1.
Harvey, N. T., Chan, J. and Wood, B. A. (2017) ‘Skin biopsy in the diagnosis of neoplastic skin disease’, Australian Family Physician, 46(5), pp. 289-294.
Hendrickx, D. A. E. et al. (2017) ‘Gene expression profiling of multiple sclerosis pathology identifies early patterns of demyelination surrounding chronic active lesions’, Frontiers in Immunology, 8 (DEC). doi: 10.3389/fimmu.2017.01810.
Hogan, J. J., Mocanu, M. and Berns, J. S. (2016) ‘The native kidney biopsy: Update and evidence for best practice’, Clinical Journal of the American Society of Nephrology, pp. 354-356. doi: 10.2215/CJN.05750515.
Hu, C. et al. (2009) ‘PPARG, KCNJ11, CDKAL1, CDKN2A-CDKN2B, IDE-KIF11-HHEX, IGF2BP2 and SLC30A8 are associated with type 2 diabetes in a chinese population’, PLOS ONE, 4(10). doi: 10.1371/journal.pone.0007643.
Ilic, D. et al. (2018) ‘Prostate cancer screening with prostate-specific antigen (PSA) test: A systematic review and meta-analysis’, BMJ (Online), 362. doi: 10.1136/bmj.k3519.
Ju, W., Smith, S. and Kretzler, M. (2012) ‘Genomic biomarkers for chronic kidney disease’, Translational Research, pp. 290-302. doi: 10.1016/j.trs1.2012.01.020.
Killock, D. (2018) ‘Diagnosis: CancerSEEK and destroy-A blood test for early cancer detection’, Nature Reviews Clinical Oncology, 13-133. doi: 10.1038/nrclinonc.2018.21.
Kim, W. J. and Lee, S. Do (2015) ‘Candidate genes for COPD: Current evidence and research’, International Journal of COPD, pp. 2249-2255. doi: 10.2147/COPD.S80227.
Kowalik, A., Kowalewska, M. and Goidi, S. (2017) ‘Current approaches for avoiding the limitations of circulating tumor cells detection methods—implications for diagnosis and treatment of patients with solid tumors’, Translational Research, pp. 58-84.e15. doi: 10.1016/j.trsl.2017.04.002.
Kuster, G. M. et al. (2020) ‘SARS-COV2: Should inhibitors of the renin-angiotensin systembe withdrawn in patients with COVID-19?’, European Heart Journal, pp. 1801-1803. doi: 10.1093/eurheartj/ehaa235.
Lucchinetti, C. F. et al. (2011) ‘Inflammatory Cortical Demyelination in Early Multiple Sclerosis’, New England Journal of Medicine, 365(23), pp. 2188-2197. doi: 10.1056/nejmoal100648.
Miller, A. B. (2003) ‘Book Review Fulfilling the Potential of Cancer Prevention and Early Detection Edited by Susan J. Curry, Tim Byers, and Maria Hewitt. 542 pp., illustrated. Washington, D.C., National Academies Press, 2003. $59.95. 0-309-08254-4’, New England Journal of Medicine, 349(18), pp. 1781-1782. doi: 10.1056/nejm200310303491824.
Mlika, M. et al. (2017) ‘Liquid biopsy in lung cancer’, Tunisie Medicale, pp. 965-971.
Monferrer, E. et al. (2019) ‘High Oct4 expression: Implications in the pathogenesis of neuroblastic tumours’, BMC Cancer, 19(1). doi: 10.1186/s12885-018-5219-3.
Riggi, N., Aguet, M. and Stamenkovic, I. (2018) ‘Cancer Metastasis: A Reappraisal of Its Underlying Mechanisms and Their Relevance to Treatment’, Annual Review of Pathology: Mechanisms of Disease, 13, pp. 117-140. doi: 10.1146/annurev-pathol-020117-044127.
Schiffman, J. D., Fisher, P. G. and Gibbs, P. (2015) ‘Early Detection of Cancer: Past, Present, and Future’, American Society of Clinical Oncology Educational Book, (35), pp. 57-65. doi: 10.14694/edbook am.2015.35.57.
Shaw, P. and Crosby, T. (2008) ‘Cancer of unknown primary’, in Practical Clinical Oncology, pp. 442-448. doi: 10.1017/CBO9780511545375.039.
Shen, Z., Wu, A. and Chen, X. (2017) ‘Current detection technologies for circulating tumor cells’, Chemical Society Reviews, pp. 2038-2056. doi: 10.1039/c6cs00803h.
Sherman, K. E. et al. (2007) ‘Liver biopsy in cirrhotic patients’, American Journal of Gastroenterology, 102(4), pp. 789-793. doi: 10.1111/j.1572-0241.2007.01110.x.
Sodja, E. et al. (2016) ‘The prognostic value of whole blood SOX2, NANOG and OCT4 mRNA expression in advanced small-cell lung cancer’, Radiology and Oncology, 50(2), pp. 188-196. doi: 10.1515/raon-2015-0027.
Temilola, D. O. et al. (2019) ‘The Prospect and Challenges to the Flow of Liquid Biopsy in Africa’, Cells. doi: 10.3390/cells8080862.
Vaduganathan, M. et al. (2020) ‘Renin-Angiotensin-Aldosterone System Inhibitors in Patients with Covid-19’, New England Journal of Medicine, 382(17), pp. 1653-1659. doi: 10.1056/nejmsr2005760.
Wang, Y. J. and Herlyn, M. (2015) ‘The emerging roles of Oct4 in tumor-initiating cells’, American Journal of Physiology—Cell Physiology, 309(11), pp. C709-C718. doi: 10.1152/ajpce11.00212.2015.
Westcott, P. M. K. and To, M. D. (2013) ‘The genetics and biology of KRAS in lung cancer’, Chinese Journal of Cancer, pp. 63-70. doi: 10.5732/cjc.012.10098.
Wong, S. Q. et al. (2014) ‘Sequence artefacts in a prospective series of formalin-fixed tumours tested for mutations in hotspot regions by massively parallel sequencing’, BMC Medical Genomics, 7(1). doi: 10.1186/1755-8794-7-23.
Zhong, L. et al. (2016) ‘Correlation between gene expression and osteoarthritis progression in human’, International Journal of Molecular Sciences, 17(7). doi: 10.3390/ijms 17071126.
Zhou, B. et al. (2020) ‘Application of exosomes as liquid biopsy in clinical diagnosis’, Signal Transduction and Targeted Therapy. doi: 10.1038/s41392-020-00258-9.

1.-21. (canceled)

22. An in-vitro method for detecting a medical condition in a subject, comprising:
(a) obtaining a blood sample from the subject;
(b) adding a salt solution to the blood sample;
(c) layering the blood sample over a neutral buffer;
(d) subjecting the blood sample to a density gradient centrifugation at a speed of between 200 g and 900 g to obtain a first pellet comprising red blood cells (RBCs);
(e) lysing the RBCs to obtain an RBC-lysed solution;
(f) centrifuging the RBC-lysed solution at a speed of between 400 g and 4000 g to obtain a second pellet;
(g) subjecting the second pellet to cell lysis and extracting ribonucleic acid (RNA) molecules from the second pellet;
(h) assaying the RNA molecules to determine an expression level of Oct 4A; and
(i) comparing the expression level of Oct 4A with a reference expression level of Oct 4A, wherein an increase of 1.1 to 3 folds in the expression level of Oct 4A as compared to the reference expression level of Oct 4A indicates a presence of the medical condition in the subject.

23. The method of claim 22, wherein the blood sample is a peripheral blood sample.

24. The method of claim 22, wherein extracting the RNA molecules comprises a technique selected from the group consisting of: guanidinium thiocyanate-phenol-chloroform nucleic acid extraction, cesium chloride gradient centrifugation, cetyltrimethylammonium bromide nucleic acid extraction, alkaline extraction, resin-based extraction, and solid phase nucleic acid extraction.

25. The method of claim 22, wherein assaying the RNA molecules comprises a technique selected from the group consisting of: quantitative polymerase chain reaction (PCR), flow cytometry, and next-generation sequencing (NGS).

26. The method of claim 22, wherein the medical condition is selected from the group consisting of multiple sclerosis, kidney disorders, skin disease, liver disease, lung disease, cardiovascular diseases, osteoarthritis, viral disease, cancer, and diabetes.

27. The method of claim 26, wherein the medical condition is cancer.

28. The method of claim 22, wherein the neutral buffer comprises Ficoll Hypaque solution.

29. The method of claim 22, wherein lysing the RBCs comprises treating the first pellet with ammonium chloride.

30. An in-vitro method for predicting onset of cancer or detecting a presence of tumor or cancer in a subject, comprising:
(a) obtaining a blood sample from the subject;
(b) adding a salt solution to the blood sample;
(c) layering the blood sample of over a neutral buffer;
(d) subjecting the blood sample to a density gradient centrifugation at a speed of between 200 g and 900 g, to obtain a first pellet comprising red blood cells (RBCs);
(e) lysing the RBCs to obtain an RBC-lysed solution;
(f) centrifuging the RBC-lysed solution at a speed of between 400 g and 4000 g to obtain a second pellet;
(g) subjecting the second pellet to cell lysis and extracting ribonucleic acid (RNA) molecules from the second pellet;
(h) assaying the RNA molecules to determine an expression level of Oct 4A; and
(i) comparing the expression level of Oct 4A with a reference expression level of Oct 4A,
wherein an increase of 3 to 5 folds in the expression level of Oct 4A as compared to the reference expression level of Oct 4A predicts the onset of cancer in the subject, and
wherein an increase of at least 5 folds in the expression level of Oct 4A as compared to the reference expression level of Oct 4A indicates a presence of tumor or cancer in the subject.

31. The method of claim 30, wherein the blood sample is a peripheral blood sample.

32. The method of claim 30, wherein extracting the RNA molecules comprises a technique selected from the group consisting of: guanidinium thiocyanate-phenol-chloroform nucleic acid extraction, cesium chloride gradient centrifugation, cetyltrimethylammonium bromide nucleic acid extraction, alkaline extraction, resin-based extraction, and solid phase nucleic acid extraction.

33. The method of claim 30, wherein assaying the RNA molecules comprises a technique selected from the group consisting of: quantitative polymerase chain reaction (PCR), flow cytometry, and next-generation sequencing (NGS).

34. The method of claim 30, wherein the neutral buffer comprises Ficoll Hypaque solution.

35. The method of claim 30, wherein lysing the RBCs comprises treating the first pellet with ammonium chloride.

36. The method of claim 30, further comprising performing a sequence-based assay on the RNA molecules to detect a mutation in at least one cancer-related marker, wherein a detected presence of the mutation in the at least one cancer-related marker indicates presence of a specific type of cancer.

37. The method of claim 36, wherein the cancer-related marker is selected from the group consisting of: ABL1, EVI1, MYC, APC, IL2, TNFAIP3, ABL2, EWSRI, MYCL1, ARHGEF12, JAK2, TP53, AKT1, FEY, MYCN, ATM, MAP2K4, TSC1, AKT2, FGFR1, NCOA4, BCL11B, MDM4, TSC2, ATF1, FGFR1OP, NFKB2, BLM, MEN1, VHL, BCL11A, FGFR2, NRAS, BMPRIA, MLH1, WRN, BCL2, FUS, NTRK1, BRCA1, MSH2, WT1, BCL3, GOLGA5, NUP214, BRCA2, NF1, BCL6, GOPC, PAX8, CARS, NF2, BCR, HMGA1, PDGFB, CBFA2T3, NOTCH1, BRAF, HMGA2, PIK3CA, CDHI, NPM1, CARD11, HRAS, PIM1, CDH11, NR4A3, CBLB, IRF4, PLAGI, CDK6, NUP98, CBLC, JUN, PPARG, CDKN2C, PALB2, CONDI, KIT, PTPN11, CEBPA, PML, CCND2, KRAS, RAF1, CHEK2, PTEN, CCND3, LCK, REL, CREB1, RB1, CDX2, LM02, RET, CREBBP, RUNX1, CTNNB1, MAF, ROS1, CYLD, SDHB, DDB2, MAFB, SMO, DDX5, SDHD, DDIT3, MAML2, SS18, EXT1, SMARCA4, DDX6, MDM2, TCLIA, EXT2, SMARCB1, DEK, MET, TET2, FBXW7, SOCS1, EGFR, MITF, TFG, FH, STK11, ELK4, MLL, TLX1, FLT3, SUFU, ERBB2, MPL, TPR, FOXP1, SUZ12, ETV4, MYB, USP6, GPC3, SYK, ETV6, IDH1, TCF3, and combinations thereof.

38. The method of claim 30, further comprising administering anti-cancer therapy to the subject for treating cancer, wherein the anti-cancer therapy decreases expression level of Oct 4A in the subject.

39. The method of claim 38, wherein the anti-cancer therapy comprises chemotherapy.

40. The method of claim 30, wherein an increase of 5 to 10 folds in the expression level of Oct 4A as compared to the reference expression level of Oct 4A indicates stage-I of cancer, wherein an increase of 10 to 15 folds in the expression level of Oct 4A as compared to the reference expression level of Oct 4A indicates stage-II of cancer, wherein an increase of 15 to 20 folds in the expression level of Oct 4A as compared to the reference expression level of Oct 4A indicates stage-III of cancer, and wherein an increase of at least 20 folds in the expression level of Oct 4A as compared to the reference expression level of Oct 4A indicates stage-IV of cancer.

41. An in-vitro method for detecting a positive response to anti-cancer therapy, comprising:
(a) obtaining a first blood sample from a subject before administration of an anti-cancer therapy;
(b) adding a salt solution to the first blood sample;
(c) layering the first blood sample over a first neutral buffer;
(d) subjecting the first blood sample to a density gradient centrifugation at a speed of between 200 g and 900 g to obtain a first pellet comprising red blood cells (RBCs);
(e) lysing the RBCs in the first pellet to obtain a first RBC-lysed solution;
(f) centrifuging the first RBC-lysed solution at a speed of between 400 g and 4000 g to obtain a second pellet;
(g) subjecting the second pellet to cell lysis and extracting first ribonucleic acid (RNA) molecules from the second pellet;
(h) assaying the first RNA molecules to determine a first expression level of Oct 4A;
(i) obtaining a second blood sample from the subject after at least 2 weeks of administration of the anti-cancer therapy;
(j) adding a salt solution to the second blood sample;
(k) layering the second blood sample over a second neutral buffer, and subjecting the second blood sample to a density gradient centrifugation at a speed of between 200 g and 900 g to obtain a third pellet comprising RBCs;
(l) lysing the RBCs in the third pellet to obtain a second RBC-lysed solution;
(m) centrifuging the second RBC-lysed solution at a speed of between 400 g and 4000 g to obtain a fourth pellet;
(n) subjecting the fourth pellet to cell lysis and extracting second ribonucleic acid (RNA) molecules from the fourth pellet;
(o) assaying the second RNA molecules to determine a second expression level of Oct 4A; and
(p) comparing the second expression level of Oct 4A with the first expression level of Oct 4A,
wherein a decrease in the second expression level of Oct 4A as compared to the first expression level of Oct 4A indicates a positive response to the anti-cancer therapy.

42. The method of claim 41, wherein the first blood sample and the second blood sample are peripheral blood samples.

43. The method of claim 41, wherein extracting the first RNA molecules and the second RNA molecules comprises a technique selected from the group consisting of: guanidinium thiocyanate-phenol-chloroform nucleic acid extraction, cesium chloride gradient centrifugation, cetyltrimethylammonium bromide nucleic acid extraction, alkaline extraction, resin-based extraction, and solid phase nucleic acid extraction.

44. The method of claim 41, wherein assaying the first RNA molecules and the second RNA molecules comprises a technique selected from the group consisting of: quantitative polymerase chain reaction (PCR), flow cytometry, and next-generation sequencing (NGS).

45. The method of claim 41, wherein the neutral buffer comprises Ficoll Hypaque solution.

46. The method of claim 41, wherein lysing the RBCs comprises treating the first pellet and the third pellet with ammonium chloride.
